(19) United States
(12) Patent Application Publication
(10) Pub. No.: US 2015/0060280 A1
(43) Pub. Date: Mar. 5, 2015

(54) METHOD FOR CLASSIFYING AND DISCRIMINATING JATROPHA LINES USING RETROTRANSPOSON AS A MARKER

(71) Applicants: SUMITOMO ELECTRIC INDUSTRIES, LTD., Osaka (JP); Osaka University, Osaka (JP)

(72) Inventors: Kiichi FUKUI, Osaka (JP); Suguru TSUCHIMOTO, Osaka (JP); Hiroe SAKAI, Osaka (JP); Atefeh ALIPOUR, Osaka (JP); Toshifumi HOSOYA, Osaka (JP); Yasuhisa YUSHIO, Osaka (JP)

(21) Appl. No.: 14/472,999

(22) Filed: Aug. 29, 2014

(30) Foreign Application Priority Data
Sep. 3, 2013 (JP) 2013-181939

(51) Int. Cl. C12Q 1/68; G01N 27/447

(52) U.S. Cl. CPC C12Q 1/6895; G01N 27/44747; C12Q 2600/13; C12Q 2600/156
USPC 204/461; 435/6.12

(57) ABSTRACT

Jatropha lines can be classified and discriminated by a method including the steps of: conducting a nucleic acid amplification reaction using a primer set including (i) adjacent forward primer and (ii) adjacent reverse primer, and (iii) LTR forward primer or LTR reverse primer, or (iv) RTP forward primer or RTP reverse primer, wherein DNA prepared from Jatropha that is an objective of determination, is used as a template; and determining the presence or absence of insertion of LTR-type retrotransposon including the retrotransposon sequence used in (iii) or (iv), on the basis of the presence or absence and length of an amplification product obtained by the amplification reaction.

### BACKGROUND OF THE INVENTION

1. Field of the Invention

The present invention relates to a method for classifying and discriminating Jatropha lines based on a LTR (Long Terminal Repeat) sequence of copia type retrotransposon that is newly identified in Jatropha genome, and a kit for classifying and discriminating lines.

2. Description of the Background Art

Jatropha curcas L attracts attentions as biological resources for production of biodiesel fuel because non-edible Jatropha oil can be produced from its seeds. For improving the availability of Jatropha as resources for biological fuel, it is necessary to select Jatropha lines that can grow even in marginal lands. In addition, for improving the resistance to environmental stress and for developing varieties of Jatropha achieving high yield of oil, it is demanded to classify and discriminate the currently existing varieties and lines of Jatropha and utilize them for breeding.

With regard to Jatropha, however, definite classification of varieties has not been made yet, and definite classification in lines has not been made yet.

Jatropha is believed to originate in Mexico and South America, and to spread to Africa and Asia. Although Mexico and Guatemala line of Jatropha is distinguished from Asia and Africa line, a method for classifying and discriminating these lines has not been established yet.

As a method for discriminating varieties of plants including classification of lines, a method for discriminating varieties based on DNA polymorphism has come into use in association with recent development in molecular biological technology. The discrimination method using a DNA marker is advantageous in that different varieties can be discriminated regardless of whether its character is expressed in the plant body.

As a DNA marker, RFLP (Restriction Fragment Length Polymorphism), AFLP (Amplified Fragment Length Polymorphism), CAPS (Cleaved Amplified Polymorphic Sequence), SSR (Simple Sequence Repeat) and so on are known.

As to Jatropha, a SSR marker is used for discriminating between Mexico and Guatemala line and Asia and Africa line.

The SSR used herein refers to a sequence in which a motif of one to four nucleotides is repeated tandem. This is suited as a DNA marker because a large number of SSRs are dispersed in genome, and the number of repetition is easily varied. Additionally, the SSR marker is codominant and has higher reliability than dominant markers such as RAPD, and thus gives more information. For example, different lines can be discriminated by different numbers of repetition.

Since polymorphism of SSR is based on a difference in the number of repetition of sequence of two to four bases, a difference in speciation such as a difference between Mexico and Guatemala line and Asia and Africa line can be detected based on the difference in the repetition number, however, further studies are required to distinguish between these lines accurately. In addition, it is difficult to distinguish and discriminate between sublines in the same line with SSR.

Besides the above, retrotransposon, which is one of repetitive sequences abundantly present in genome of plants, is also known as an excellent DNA marker. When the sequence of retrotransposon in genome is transcribed, the transcript is reverse-transcribed and a copy DNA fragment of the original sequence is generated. As the DNA fragment is newly inserted into genome, the inserted retrotransposon sequence is stably inherited to progeny. Therefore, a genome sequence adjacent to retrotransposon is inherent to an individual variety or line, and thus polymorph in a variety or polymorph in a line exists such that at a certain genome position where retrotransposon is inserted in one variety or line, insertion is not observed in another variety or line. Thus, retrotransposon is an excellent codominant genetic marker because a large number of retrotransposons are dispersed in plant genome, and the site of insertion is specific to an individual variety or line. In addition, the presence or absence of insertion of retrotransposon in genome elucidates the course of evolution, and it is possible to know the difference between lines more accurately than by SSR.

As a method for discriminating varieties using a retrotransposon marker, Japanese Patent No. 4889328 proposes discriminating varieties of chrysanthemum by using a primer set including a primer designed in a retrotransposon sequence, as a method for discriminating varieties of chrysanthemum.

Japanese Patent Laying-Open No. 2006-42808 and Japanese Patent Laying-Open No. 2010-172322 disclose a method of detecting whether or not retrotransposon is inserted at a specific position of plant genome, as a method for determining a plant species of a material plant included in a processed food.

Andrew J. Flavell et al., “Retrotransposon-based insertion polymorphisms (RBIP) for high throughput marker analysis”, The Plant Journal, (1998) 16(5), p. 643-650 discloses discriminating varieties of bean (Pisum sativum) according to the presence or absence of insertion of LTR-type retrotransposon.

### SUMMARY OF THE INVENTION

Regarding Jatropha, a difference between Mexico and Guatemala line and Asia and Africa line, and a difference in Mexico and Guatemala line have been detected by using a SSR marker, however, there is no particular report describing that Asia and Africa line is further classified and discriminated with a reliable marker, and there is no particular report about a useful marker.

It is an object of the present invention to provide a method for classifying and discriminating Jatropha lines using a DNA marker capable of classifying and discriminating Jatropha lines, and a primer kit for the classification and discrimination.

The present inventors tried a method of using a retrotransposon marker as a method for not only distinction between Mexico and Guatemala line and Asia and Africa line, but also for classifying and discriminating sublines in these lines.

Regarding Jatropha, identification information about a part of a protein-coding sequence is open to public. Unidentified sequence data is open to public as a part of a genomic sequence (http://www.kazusa.or.jp/jatropha).

The present inventors identified the sequences of major members of Ty1-copia type retrotransposon and LTRs thereof on the basis of the aforementioned sequence data, and designed primers capable of detecting the presence or absence of insertion of retrotransposon in a specified region on the basis of the identified LTR sequences, thereby completing the present invention.

That is, the method of the present invention for classifying and discriminating Jatropha lines using retrotransposon as a marker, is a method for classifying and discriminating Jatropha lines using a LTR (Long Terminal Repeat)-type retrotransposon marker, including the steps of:

conducting a nucleic acid amplification reaction using a primer set including primers of the following (i) and (ii), and a primer of the following (iii) or (iv), wherein DNA prepared from Jatropha that is an objective of determination, is used as a template:

(i) a forward primer of 15 to 60 bases having complementarity with a base sequence of 5′-side adjacent region of left LTR (hereinafter, referred to as “adjacent forward primer”),

(ii) a reverse primer of 15 to 60 bases having complementarity with a base sequence of 3′-side adjacent region of right LTR (hereinafter, referred to as “adjacent reverse primer”),

(iii) a forward primer of 15 to 60 bases having complementarity with 3′-terminal sequence of the right LTR sequence and its 3′-side adjacent sequence, or a reverse primer of 15 to 60 bases having complementarity with 5′-terminal sequence of the left LTR sequence and its 5′-side adjacent sequence (hereinafter, referred to as “LTR primer”), and

(iv) a forward or reverse primer of 15 to 60 bases having complementarity with at least a part of a retrotransposon sequence existing between the left and right LTR sequences (hereafter, referred to as “RTP forward primer” or “RTP reverse primer”); and

determining the presence or absence of insertion of LTR-type retrotransposon including the retrotransposon sequence used in (iii) or (iv), on the basis of the presence or absence and length of an amplification product obtained in the amplification reaction.

The present invention also encompasses a primer set that is effective for performing the discrimination method of the present invention. The kit for classifying and discriminating Jatropha lines of the present invention is a kit for classifying and discriminating Jatropha lines including at least one primer set selected from the following primer sets:

(a) a primer set of at least one primer selected from the group consisting of primers of SEQ ID NOs: 1, 2 and 3, and primers having a sequence identity of greater than or equal to 80% in 15 bases on the 3′-end side of said respective primers;

(b) a primer set of at least two primers selected from the group consisting of primers of SEQ ID NOs: 4, 5 and 6, and primers having a sequence identity of greater than or equal to 80% in 15 bases on the 3′-end side of said respective primers;

(c) a primer set of at least two primers selected from the group consisting of primers of SEQ ID NOs: 7, 8, 9 and 10, and primers having a sequence identity of greater than or equal to 80% in 15 bases on the 3′-end side of said respective primers;

(d) a primer set of at least two primers selected from the group consisting of primers of SEQ ID NOs: 11, 12, 13 and 14, and primers having a sequence identity of greater than or equal to 80% in 15 bases on the 3′-end side of said respective primers;

(e) a primer set of at least two primers selected from the group consisting of primers of SEQ ID NOs: 15, 16, 17 and 18, and primers having a sequence identity of greater than or equal to 80% in 15 bases on the 3′-end side of said respective primers;

(f) a primer set of at least two primers selected from the group consisting of primers of SEQ ID NOs: 19, 20, 21 and 22, and primers having a sequence identity of greater than or equal to 80% in 15 bases on the 3′-end side of said respective primers;

(g) a primer set of at least two primers selected from the group consisting of primers of SEQ ID NOs: 23, 24, 25 and 26, and primers having a sequence identity of greater than or equal to 80% in 15 bases on the 3′-end side of said respective primers;

(h) a primer set of at least two primers selected from the group consisting of primers of SEQ ID NOs: 27, 28, 29 and 30, and primers having a sequence identity of greater than or equal to 80% in 15 bases on the 3′-end side of said respective primers;

(i) a primer set of at least two primers selected from the group consisting of primers of SEQ ID NOs: 31, 32, 33 and 34, and primers having a sequence identity of greater than or equal to 80% in 15 bases on the 3′-end side of said respective primers; and

(j) a primer set of at least one primer selected from the group consisting of primers of SEQ ID NOs: 35, 36, 37 and 38, and primers having a sequence identity of greater than or equal to 80% in 15 bases on the 3′-end side of said respective primers.

In this specification, “right” and “left” respectively refer to right (5′-side) and left (3′-side) in a genome map expressed so that bases are arranged from 5′-side to 3′-side according to the custom.

According to the method of the present invention for classifying and discriminating Jatropha lines using retrotransposon as a marker, Jatropha lines, which have not been clearly classified and discriminated yet, can be classified in consideration of the course of evolution and speciation, and a genealogical chart can be established.

### BRIEF DESCRIPTION OF THE DRAWINGS

FIG. 1 is a chart for describing the structure of copia type LTR retrotransposon.

FIG. 2 is a chart showing a sequence of retrotransposon of Jc3 family.

FIG. 3 is a chart showing a sequence of retrotransposon of Jc7 family.

FIG. 4 is a chart showing a sequence of retrotransposon of Jc8 family.

FIG. 5 is a chart showing a sequence of retrotransposon of Jc9 family.

FIG. 6 is a chart for describing a primer for use in the method for classifying and discriminating lines of the present invention.

FIG. 7 is a chart for describing an embodiment of the method for classifying and discriminating lines.

FIG. 8 is a gel image of the electrophoretic result obtained in Example.

### DESCRIPTION OF THE PREFERRED EMBODIMENTS

Hereinafter, embodiments of the present invention will be described, and it is to be noted that the embodiments disclosed herein are given for exemplification but not for limitation in any points. The scope of the present invention is specified by claims, and every modification within the equivalent meaning and the scope of claims is intended to be included.

The method of the present invention for classifying and discriminating Jatropha lines of the present embodiment is a method for classifying and discriminating Jatropha lines using a LTR (Long Terminal Repeat)-type retrotransposon marker, including the steps of:

conducting a nucleic acid amplification reaction using a primer set including primers of the following (i) and (ii), and a primer of the following (iii) or (iv), wherein DNA prepared from Jatropha that is an objective of determination, is used as a template:

(i) a forward primer of 15 to 60 bases having complementarity with a base sequence of 5′-side adjacent region of left LTR (hereinafter, referred to as “adjacent forward primer”),

(ii) a reverse primer of 15 to 60 bases having complementarity with a base sequence of 3′-side adjacent region of right LTR (hereinafter, referred to as “adjacent reverse primer”),

(iii) a forward primer of 15 to 60 bases having complementarity with 3′-terminal sequence of the right LTR sequence and its 3′-side adjacent sequence, or a reverse primer of 15 to 60 bases having complementarity with 5′-terminal sequence of the left LTR sequence and its 5′-side adjacent sequence (hereinafter, referred to as “LTR primer”), and

(iv) a forward or reverse primer of 15 to 60 bases having complementarity with at least a part of a retrotransposon sequence existing between the left and right LTR sequences (hereafter, referred to as “RTP forward primer” or “RTP reverse primer”); and

determining the presence or absence of insertion of LTR-type retrotransposon including the retrotransposon sequence used in (iii) or (iv), on the basis of the presence or absence and length of an amplification product obtained in the amplification reaction.

By using retrotransposon as a marker, it is possible to classify Jatropha lines in consideration of the course of evolution.

In addition, according to the method of the above embodiment, on the basis of the presence or absence of insertion of retrotransposon, an amplification product is obtained or not obtained, or the fragment length of the obtained amplification product varies. Therefore, it is possible to detect the presence or absence of insertion of retrotransposon on the basis of the presence or absence of an amplification product and the variation in fragment length, and to facilitate the operation.

As the primer set, it is preferred to use the (i) and (ii), and the LTR forward primer and the LTR reverse primer of (iii). This makes it possible to classify and discriminate lines that further speciate, including polymorphs of retrotransposon and polymorphs of adjacent regions.

As the retrotransposon, typically, one selected from the group consisting of retrotransposon represented by Jc3 (SEQ ID NO: 49), Jc7 (SEQ ID NO: 50), Jc8 (SEQ ID NO: 51), and Jc9 (SEQ ID NO: 52), and sequences respectively having a homology of greater than or equal to 85% with said respective sequences can be recited.

As the base sequence of LTR, a sequence represented by one selected from SEQ ID NOs: 39 to 41 and sequences respectively having a sequence identity of greater than or equal to 85% with said respective sequences can be recited.

One preferred embodiment is a method for classifying and discriminating Jatropha lines by detecting the presence or absence of LTR-type retrotransposon of Jc3S using a primer set including primers represented by SEQ ID NOs: 1, 2 and 3, or primers having a sequence identity of greater than or equal to 80% in 15 bases on the 3′-end side of said respective primers. Another preferred embodiment is a method for classifying and discriminating Jatropha lines by detecting the presence or absence of LTR-type retrotransposon of Jc9S using a primer set including primers represented by SEQ ID NOs: 35, 36, 37 and 38, or primers having a sequence identity of greater than or equal to 80% in 15 bases on the 3′-end side of said respective primers.

LTR-type retrotransposon of Jc3S and LTR-type retrotransposon of Jc9S are variants of Jc3 retrotransposon and Jc9 retrotransposon, respectively, and are retrotransposons having only a LTR sequence. Therefore, by detecting the presence or absence of the LTR sequence of Jc3S or Jc9S, it is possible to classify Jatropha lines.

As the primer set, a plurality of primer sets may be used for determining the presence or absence of insertion of a plurality of retrotransposons, and the step of classifying and discriminating Jatropha lines according to the presence or absence of insertion of each retrotransposon may be further included.

By detecting the presence or absence of insertion of a plurality of retrotransposons, and combining the detection results, it is possible to collectively classify downstream lines.

The fragment length of an amplification product of the line including retrotransposon is preferably 50 to 2000 bp. The nucleic acid amplification reaction is preferably a polymerase chain reaction (PCR). The length of the amplification product is preferably distinguished by agarose gel electrophoresis or by polyacrylamide gel electrophoresis.

By using these techniques, the detecting operation can be simplified.

In the above embodiment, concrete modes of the primer sets based on the following LTR sequences are as follows:

(1) when the LTR is the sequence of SEQ ID NO: 39 or has a homology of greater than or equal to 85% with the sequence, (a) a primer set of at least one primer selected from the group consisting of primers of SEQ ID NOs: 1, 2 and 3;

(2) when the LTR is the sequence of SEQ ID NO: 40 or has a homology of greater than or equal to 85% with the sequence, (b) a primer set of at least two primers selected from the group consisting of primers of SEQ ID NOs: 4, 5 and 6;

(3) when the LTR is the sequence of SEQ ID NO: 41 or has a homology of greater than or equal to 85% with the sequence, (c) a primer set of at least two primers selected from the group consisting of primers of SEQ ID NOs: 7, 8, 9 and 10;

(4) when the LTR is the sequence of SEQ ID NO: 42 or has a homology of greater than or equal to 85% with the sequence, (d) a primer set of at least two primers selected from the group consisting of primers of SEQ ID NOs: 11, 12, 13 and 14, (5) when the LTR is the sequence of SEQ ID NO: 43 or has a homology of greater than or equal to 85% with the sequence, (e) a primer set of at least two primers selected from the group consisting of primers of SEQ ID NOs: 15, 16, 17 and 18;

(6) when the LTR is the sequence of SEQ ID NO: 44 or has a homology of greater than or equal to 85% with the sequence, (f) a primer set of at least two primers selected from the group consisting of primers of SEQ ID NOs: 19, 20, 21 and 22;

(7) when the LTR is the sequence of SEQ ID NO: 45 or has a homology of greater than or equal to 85% with the sequence, (g) a primer set of at least two primers selected from the group consisting of primers of SEQ ID NOs: 23, 24, 25 and 26;

(8) when the LTR is the sequence of SEQ ID NO: 46 or has a homology of greater than or equal to 85% with the sequence, (h) a primer set of at least two primers selected from the group consisting of primers of SEQ ID NOs: 27, 28, 29 and 30;

(9) when the LTR is the sequence of SEQ ID NO: 47 or has a homology of greater than or equal to 85% with the sequence, (i) a primer set of at least two primers selected from the group consisting of primers of SEQ ID NOs: 31, 32, 33 and 34; and

(10) when the LTR is the sequence of SEQ ID NO: 48 or has a homology of greater than or equal to 85% with the sequence, (j) a primer set of at least one primer selected from the group consisting of primers of SEQ ID NOs: 35, 36, 37 and 38.

The present invention also encompasses a kit for classifying and discriminating Jatropha lines for executing the embodiment of the present invention. As a preferred embodiment, a kit for classifying and discriminating Jatropha lines including at least one primer set selected from the following primer sets can be recited:

(a) a primer set of at least one primer selected from the group consisting of primers of SEQ ID NOs: 1, 2 and 3, and primers having a sequence identity of greater than or equal to 80% in 15 bases on the 3′-end side of said respective primers;

(b) a primer set of at least two primers selected from the group consisting of primers of SEQ ID NOs: 4, 5 and 6, and primers having a sequence identity of greater than or equal to 80% in 15 bases on the 3′-end side of said respective primers;

(c) a primer set of at least two primers selected from the group consisting of primers of SEQ ID NOs: 7, 8, 9 and 10, and primers having a sequence identity of greater than or equal to 80% in 15 bases on the 3′-end side of said respective primers;

(d) a primer set of at least two primers selected from the group consisting of primers of SEQ ID NOs: 11, 12, 13 and 14, and primers having a sequence identity of greater than or equal to 80% in 15 bases on the 3′-end side of said respective primers;

(e) a primer set of at least two primers selected from the group consisting of primers of SEQ ID NOs: 15, 16, 17 and 18, and primers having a sequence identity of greater than or equal to 80% in 15 bases on the 3′-end side of said respective primers;

(f) a primer set of at least two primers selected from the group consisting of primers of SEQ ID NOs: 19, 20, 21 and 22, and primers having a sequence identity of greater than or equal to 80% in 15 bases on the 3′-end side of said respective primers;

(g) a primer set of at least two primers selected from the group consisting of primers of SEQ ID NOs: 23, 24, 25 and 26, and primers having a sequence identity of greater than or equal to 80% in 15 bases on the 3′-end side of said respective primers;

(h) a primer set of at least two primers selected from the group consisting of primers of SEQ ID NOs: 27, 28, 29 and 30, and primers having a sequence identity of greater than or equal to 80% in 15 bases on the 3′-end side of said respective primers;

(i) a primer set of at least two primers selected from the group consisting of primers of SEQ ID NOs: 31, 32, 33 and 34, and primers having a sequence identity of greater than or equal to 80% in 15 bases on the 3′-end side of said respective primers; and

(j) a primer set of at least one primer selected from the group consisting of primers of SEQ ID NOs: 35, 36, 37 and 38, and primers having a sequence identity of greater than or equal to 80% in 15 bases on the 3′-end side of said respective primers.

The above primer sets may be used alone or in combination of a plurality of kinds, or may include all of (a) to (j).

### <Retrotransposon>

In the method for classifying and discriminating Jatropha lines of the present invention, the retrotransposon used as a DNA marker is LTR-type retrotransposon having a repetitive sequence called LTR (Long Terminal Repeat) on both terminals of the sequence. Among retrotransposons, in LTR-type retrotransposon classified into Ty1-copia type retrotransposon, as shown in FIG. 1, LTR is present on its both ends, and PBS (primer binding site) and PPT (poly purine truct), which are sequences required in reverse transcription, are present directly inside the LTRs of 5′-side and 3′-side, respectively. In the internal translation region sandwiched by LTRs, there are a pol region coding a protein required for formation and incorporation of cDNA after transcription of itself, a GAG region required for forming viral particles encapsulating incorporated enzyme and the like, an integrase region, and so on.

From these features, even for a crop for which a full-length sequence of retrotransposon has not been examined, by identifying a sequence known in DNA database or by newly identifying a sequence of reverse transcription, it is possible to identify up to the terminal LTR sequences by TAIL (Thermal Asymmetric InterLaced) PCR (Polymerase Chain Reaction), Suppression PCR and the like for isolating an extended sequence thereof.

In the case of Jatropha, about 32000 copia type retrotransposons are known to exist. The present inventors aligned Contig sequences on the basis of data of a genome sequence whose function has not been identified for Jatropha, and identified major families of Ty1-copia type retrotransposons.

Further, the present inventors identified LTR sequences of retrotransposon. LTR sequences are substantially completely coincident sequences that are present on left and right ends of retrotransposon, and the position where homology among the retrotransposon sequences belonging to the same family disappears is defined as a terminal of LTR. Further, whether characteristic sequences (TGT at 5′-end, ACA at 3′-end) are present at ends of LTR, and whether a sequence of PBS is included in its 3′-side internal region and whether a sequence of PPT is included in its 5′-side internal region were examined. When these sequences were included, the region was determined as LTR. Then, the sequence between TGT at 5′-end and ACA at 3′-end was identified as a LTR sequence.

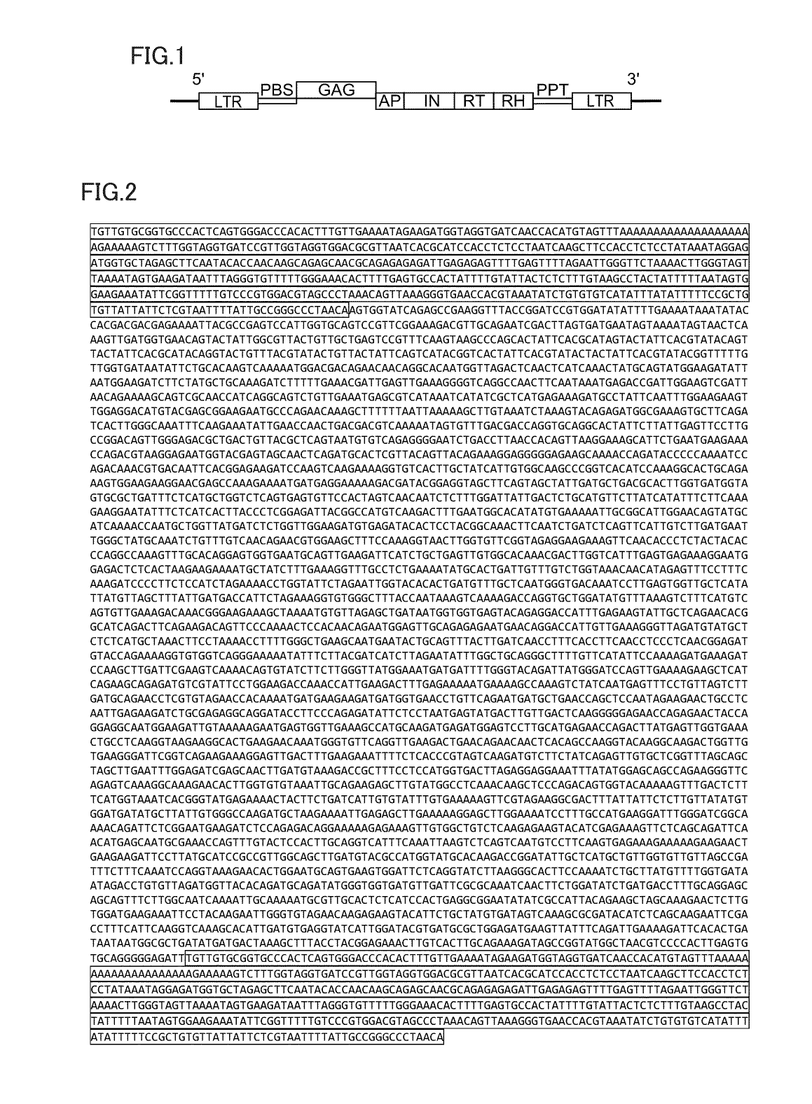

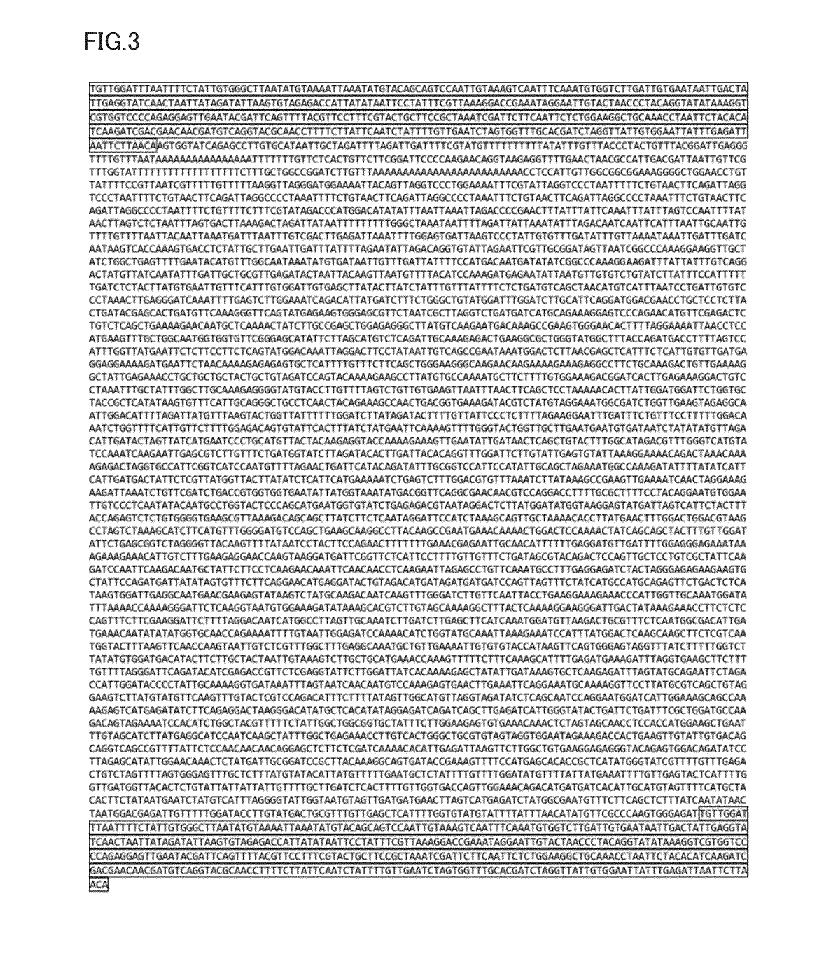

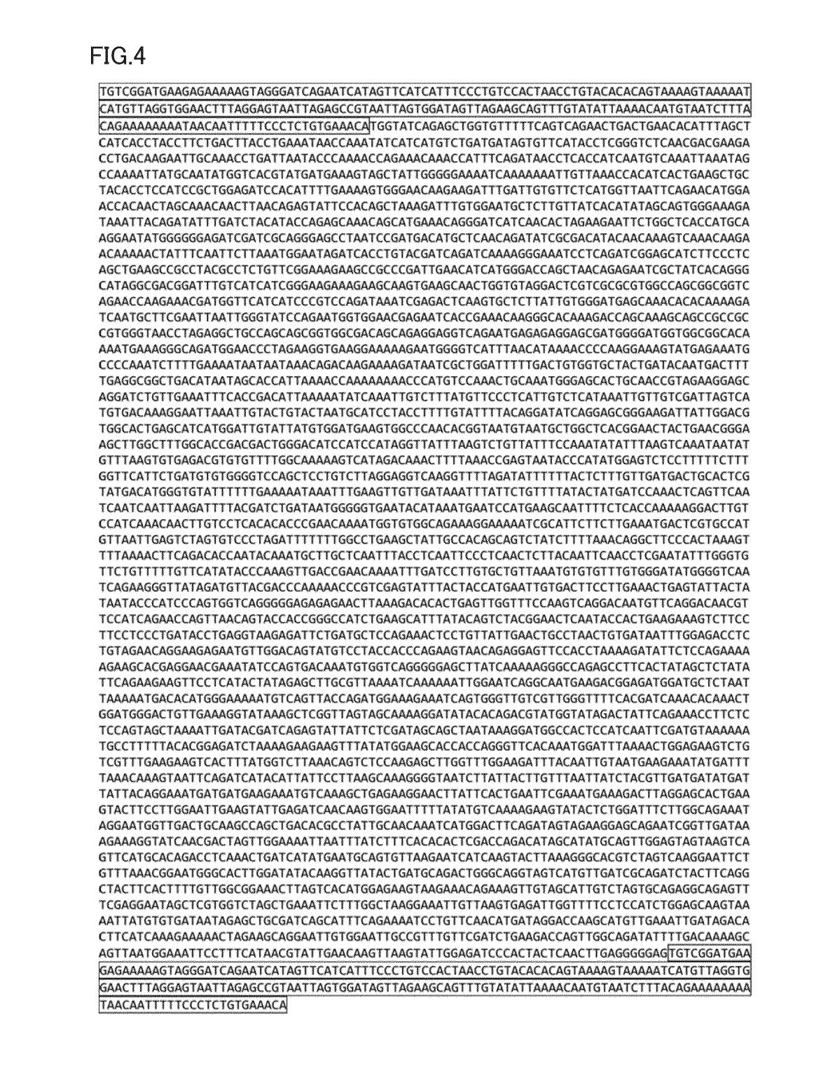

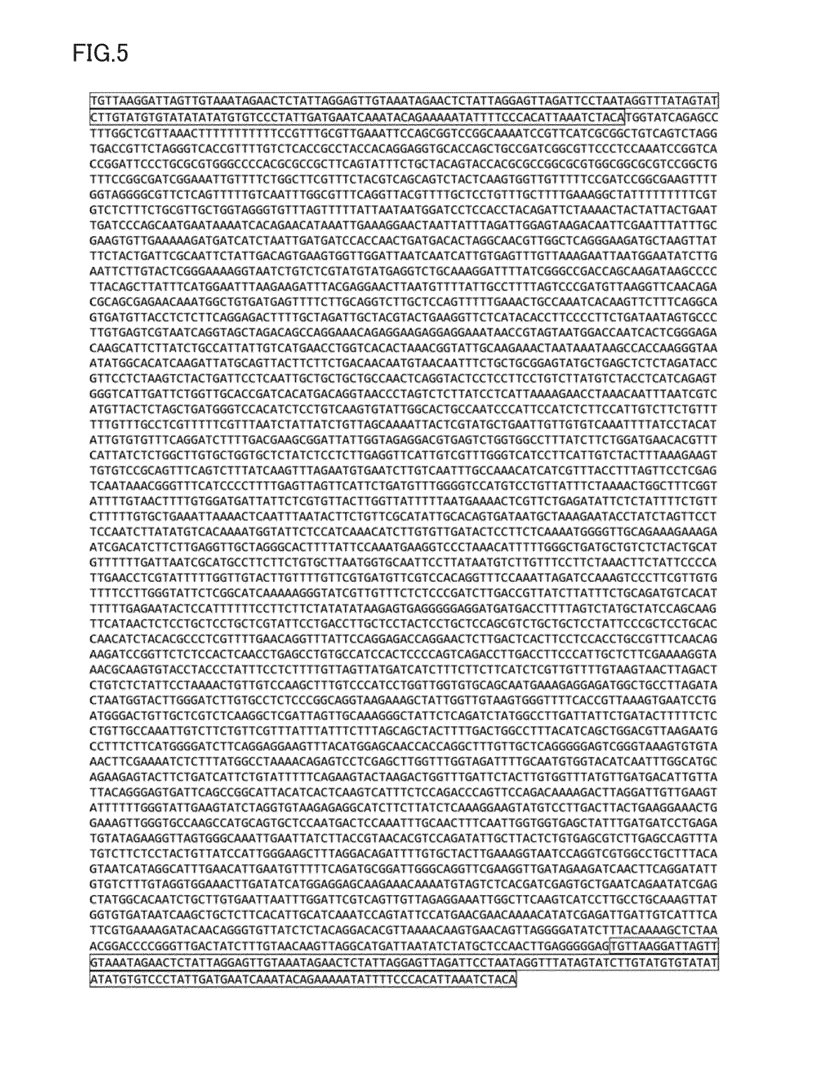

Consensus sequences of the retrotransposon families of Jatropha identified in this study are shown in FIGS. 2 to 5, and in SEQ ID NOs: 49 to 52. In FIGS. 2 to 5, the part surrounded by the square is a LTR sequence part.

The identified LTR sequences are shown in Table 1 and SEQ ID NOs: 39 to 48. Table 1-1 illustrates a LTR sequence of Jc3 family. Table 1-2 illustrates LTR sequences of Jc7 family. Jc7 family included three polymorphs (Jc7A, Jc7B, Jc7C). Table 1-3 illustrates LTR sequences of Jc8 family. Jc8 family included four polymorphs (Jc8A, Jc8B, Jc8C, Jc8D). Table 1-4 illustrates polymorphs of Jc9 family. Jc9 family included two polymorphs (Jc9A, Jc9S1). The LTR sequences shown in Table 1 are sequences of the retrotransposon families identified in this study. In actual Jatropha, these sequences include polymorphs. Usually, polymorphs having a homology of greater than or equal to 85%, preferably greater than or equal to 90% with the sequences shown in Table 1 exist.

| | | |
|---|---|---|
| Name | LTR sequence | SEQ ID NO. |

Table 1-1

| | | |
|---|---|---|
| Jc3S1_ | TGTTGTGCGGTGCCCACTCAGTGGGACCCACACTTTGTTGAAAATAAATAATGGTAGGTAATCAACCA | 39 |
| LTR | CACGTAGTTGAAAAGAAAAAAAAAAAAAAAAGAAGAAAATTGGAGAAAGTCTTTGGTAGGTGATCTG | |
| | CTGGTAGGTGGATGCGTTAATCACGCATCCATCTCTCCTCTAATCAAGCTTCCACATCTCCTATAAATA | |
| | GGAGATGGTGCTAGAGCTTCAATGCACCAACACGCAGAGCGACACAGAGTAAGATTGAGAGAGTTTT | |
| | AAGTTTTAGAATTGGGTTCTAAAACTTGGGTAGTTAAAATAGTGAAGATAATTTAGGGTGTTTTTGGG | |
| | AAACCCGTGTGAGTGACACTATTTGTATATTACTCTCTTTGTACGCCTACTATTTTTAAATAGTGGAAG | |
| | AATTATTCGGTTTTTGTCCCGTGGACGTAGCCCTAAACAGTTAAGGGTGAACCACGTAAATATCTGTG | |
| | TGTCATATTTATTTTTCTGCTGTGTTATTATTCTCGTAATTTTATTGCCGGGGCCTAACA | |

Table 1-2

| | | |
|---|---|---|
| Jc7A_ | TGTTGGATTTAATTTTCTATTGTGGGCTTAATATGTAAAATTAAATATGTACAGCAGTCTAATTGTAAAGTCAATT | 40 |
| LTR | TCAAATGTGGTCTTGATTGTGAATAATTGACTATTGAGGTATCAACTAATTATAGATATTAAGTGTAGAGACCATT | |
| | ATATAATTCCTATTTCGTTAAAGGACCGAAATAGGAATTGTACTAACCCTACAGGTATATAAAGGTCGTGGTCCCC | |
| | AGAGGAGTTGAATACGATTCAGTTTTACGTTCCTTTCGTATTGCTTCCGCTAAATCGATTCTTCAATTCTCTGGAA | |
| | GGCTGCAAACCTAATTCTATACATCAAGATCGACGAACAACGATGTCAGGTACGCAACCTTTTCTTATTCAATCTA | |
| | TTTTGTTGAATCTAGTGGTTTGCACGATCTAGGTTATTGTGGAATTATTTGAGATTAATTCTTACA | |
| Jc7B_ | TGTTGGATTTAATTTTCTATTGTGGGCTTAATATGTAAAATTAAATATGTACAGCAGTCCAATTGTAAAGTCAATT | 41 |
| LTR | TCAAATGTGGTCTTGATTGTGAATAATTGACTATTGAGGTATCAACTAATTATAGATATTAAGTGTAGAGACCATT | |
| | TATATAATCCTATTTCGTTAAAGGACCGAAATAGGAATTGTACTAACCCTACAGGTATATAAAGGTCGTGGTCCCC | |
| | AGAGGAGTTGAATACGATTCAGTTTTACGTTCCTTTCGTACTGCTTCCGCAAAATCGATTCTTCAATTCTCTGGAA | |
| | AGGCTGCAAACCTATTCTACACATCAAGATCGACGAACAACGATGTCAGGTACGCAACCTTTTCTTATTCAATCTA | |
| | TTTTGTTGAATCTAGTGATTTGCACGATCTAGGTTATTGTGGAATTATTTGAGATTAATTCTTAACA | |
| Jc7C_ | TGTTGGATTTAATTTTCTATTGTGGGCTTAATATGTAAAATTAAATATGTACAGCAGTCTAATTGTAAAGTCAATT | 42 |
| LTR | TCAAATGTGGTCTTGATTGTGAATAATTGACTATTGAGGTATCAACTAATTATAGATATTAAGTGTAGAGACCATT | |
| | ATATAATTCCTATTTCGTTAAAGGACCGAAATAGGAATTGTACTAACCCTACAGGTATATAAAGGTCGTGGTCCCC | |
| | AGAGGAGTTGAATACGATTCAGTTTTACGTTCCTTTCGTATTGCTTCCGCTAAATCGATTCTTCAATTCTCTGGAA | |
| | GGCTGCAAACCTAATTCTATACATCAAGATCGACGAACAACGATGTCAGGTACGCAACCTTTTCTTATTCAATCTA | |
| | TTTTGTTGAATCTAGTGGTTTGCACGATCTAGGTTATTATTGAATTATTTGAAATTAATTCTTAACA | |

Table 1-3

| | | |
|---|---|---|
| Jc8A_ | TGTCGGATGAAGAGAAAAAGTAGGGATCAGAATCATAGTTCATCATTTCCCTGTCCACTAACCTGTACACACAGTA | 43 |
| LTR | AAAGTAAAAATCATGTTAGGTGGAACTTTAGGAGTAATTAGAGCCGTAATTAGTGGATAGTTAGAAGCAGTTTGTA | |
| | TATTAAAACAATGTAATCTTTACAGAAAAAGAAGAACAATTTTTCCCTCTGTGAAACA | |
| Jc8B_ | TGTCGGATGAAGAGAAAAAGTAGGGATCAGAATCATAGTTCATCATTTCCCTGTCCACTAAACTGTACACACAGTA | 44 |
| LTR | AAAGTAAAAATCATGTTAGGTGGAACTTTAGGAGTAATTAGAGCCGTAATTAGTGGATAGTTAGAAGCAGTTTGTA | |
| | TATTAAAACAATGTAATCTTTACAGAAAATAATAACAATTTTTCCCTCTGTGAAACA | |
| Jc8C_ | TGTCGGATGAAGAGAAAAAGTAGGGATCAGAATCATAGTTCATCATTTCTCTGTCCACTAACCTGTACACACAGTA | 45 |
| LTR | AAAGTAAAAGTCAATGTTAGGTGGAACTTTAGGAGCAATTAGAGCCGTAATTAGTGGATAGTTAGAAGCAGTTTGT | |
| | ATATTAAAAACAATGTAATCTTTACAGAAGAAATATAACAATTTTTCTCTCTGTAAAACA | |
| Jc8D_ | TGTCGGATGAAGAGAAAAAGTAGGGATCAGAATCATAGTTCATCATTTCCCTGTCAACTAACCTGTACACACAGTA | 46 |
| LTR | AAAGTAAAAATCATGTTAGGTGGAACTTTAGGAGTAATTAGAGCCGTAATTAGTGGATAGTTAGAAGCAGTTTGTA | |
| | TATTAAAACAATGTAATCTTTACAGAAAAAAAAGAACAATTTTTCCCTCTGTGAAACA | |

Table 1-4

| | | |
|---|---|---|
| Jc9A_ | TGTTAAGGATTAGTTGTAAATAGAACTCTATTAGGAGTTGTAAATAGAACTCTATTAGGAGTTAGATTCTTAATAG | 47 |
| LTR | GTTTATAGTATCTTGTATGTGTATATATATGTGTCCCTATTGATGAATCAAATACAGAAAAATATTTTCCCACATT | |
| | AAATCTACA | |
| Jc9S2_ | TGTTAAGGATTAGTTGTAAATAGAACTCTATTAGGAGTTGTAAATAGAACTCTATTAGGAGTTAGATTCTAAATAG | 48 |
| LTR | GTTTATAGTATCTTGTATGTGTATATATATGTGTCCCTATTGATGAATCAAATATAGAAAAATATTTTCCCACATT | |
| | AAATCTACA | |

Among the retrotransposon families identified in this study, existence of a special member was identified in the lines having a common LTR. The special member has special short retrotransposon having only a substantially single LTR sequence as a result of falling of internal sequences including the PBS sequence and the PPT sequence due to homologous recombination between the left and the right LTRs in the course of evolution.

In Table 1, Jc3S1 and Jc9S2 represent LTR sequences of the members having only a single LTR sequence.

The left and the right LTRs of LTR-type retrotransposon have a completely identical sequence at the time of transcription of retrotransposon, but the left and the right sequences differ as a result of accumulation of point mutations as time passes. Therefore, by using such LTR-type retrotransposon as a DNA marker to detect the presence or absence of insertion of the retrotransposon, it is possible to know the time when the retrotransposon transferred, and it is possible to estimate the branching time on the basis of the phylogenic tree representing the course of evolution. In addition, by finding such polymorphs due to mutation of LTR sequence after transferring, polymorphs of the internal sequence, and polymorphs of the adjacent region, it is possible to classify and discriminating the closely related lines more finely.

### <Designing of Primers>

The primers used in the method of the present invention are (i), (ii), (iii), and (iv) below:

(i) a forward primer of 15 to 60 bases having complementarity with a base sequence of 5′-side adjacent region of left LTR (hereinafter, referred to as “adjacent forward primer” or “F_L primer”);

(ii) a reverse primer of 15 to 60 bases having complementarity with a base sequence of 3′-side adjacent region of right LTR (hereinafter, referred to as “adjacent reverse primer” or “F_R primer”),

(iii) a forward primer of 15 to 60 bases having complementarity with 3′-terminal sequence of the right LTR sequence and its 3′-side adjacent sequence, or a reverse primer of 15 to 60 bases having complementarity with 5′-terminal sequence of the left LTR sequence and its 5′-side adjacent sequence (hereinafter, referred to as “LTR primer”, or “LTR_R primer” and “LTR_L primer”, respectively, when the forward primer and the reverse primer are distinguished from each other), and

(iv) a forward or reverse primer of 15 to 60 bases having complementarity with at least a part of a retrotransposon sequence existing between the left and right LTR sequences (hereafter, referred to as “RTP primer”).

The above primers are used as a primer set including a combination of primers of (i), (ii), and (iii) (the discrimination method of a first embodiment); a primer set including a combination of primers of (i), (ii), and (iv) (the discrimination method of a second embodiment); a primer set including a combination of primers of LTR_L and LTR_R of (i), (ii), and (iii) (the discrimination method of a third embodiment); or a primer set including a combination of primers of (i), (ii), (iii), and (iv) (the discrimination method of a fourth embodiment).

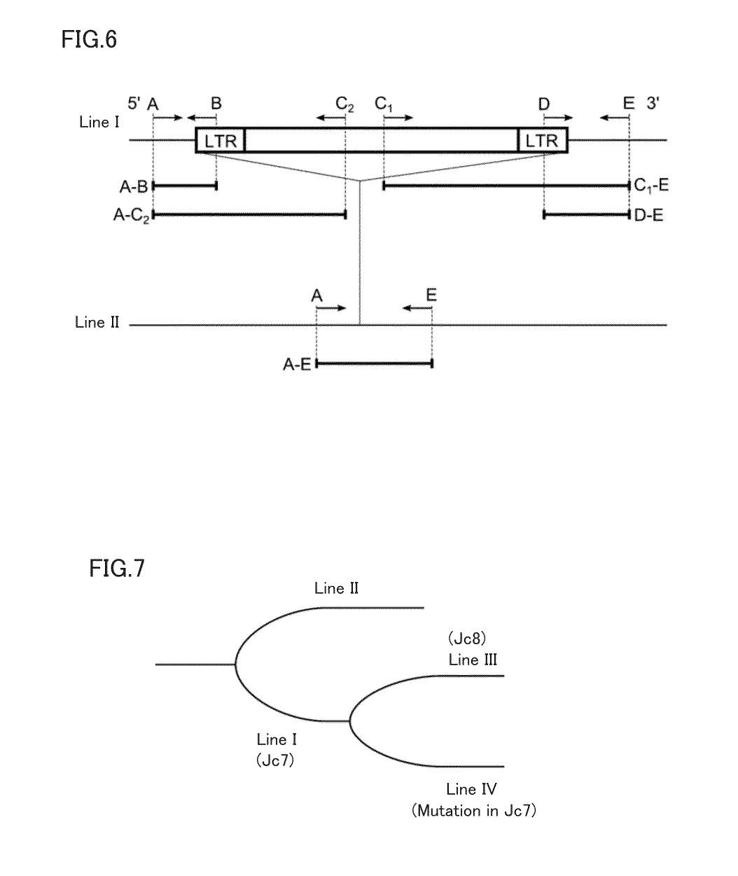

The primer of (i) group (adjacent forward primer) is a DNA fragment having 5′-end at A in the genome chart shown in FIG. 6. The primer of (ii) group (adjacent reverse primer) is a DNA fragment having 5′-end at E in the genome chart shown in FIG. 6.

The primer of (iv) group (RTP primer) is a DNA fragment having 5′-end at C₁ (forward primer) or a DNA fragment having 5′-end at C₂ (reverse primer) in the genome chart shown in FIG. 6.

In the case of the RTP primer, it is necessary to select the base positions of the starting points C₁ and C₂ so that a sequence specific to retrotransposon is included.

The primer of (iii) group (LTR primer) is a DNA fragment having 5′-end at B (reverse primer) or a DNA fragment having 5′-end at D (forward primer) in the genome chart shown in FIG. 6.

In the case of the LTR primer, it is necessary to select the base positions of the starting points B and D so that a sequence having complementarity with the LTR sequence and a part of the adjacent region and traversing the boundary of the LTR and the adjacent region is included.

Any primers have a fragment length of 15 to 60 bp, preferably 20 to 30 bp. If the fragment length is too short, specificity to the target region decreases, and a desired amplification reaction cannot be achieved. On the other hand, if the fragment length is too long, the part that is extended from the target region-specific part as a template is reduced in relation to the stably obtained amplification product, which may cause difficulty in classification and discrimination of lines.

In the primers of (i) to (iv), “having complementarity” is not limited to 100% perfect complementarity, but encompasses the complementarity of a degree such that hybridization under a stringent condition is possible.

Therefore, in designing primers, the primer may have a sequence identity of typically greater than or equal to 80%, preferably greater than or equal to 90%, and more preferably greater than or equal to 95% with the target sequence in 15 bases on the 3′-end side. Such a sequence identity makes it possible to amplify the polymorphs in which only several bases are different from each other.

Such a primer set is preferably designed so that different lengths of PCR products are obtained by the nucleic acid amplification reaction. Preferably, the primer set is designed so that the fragment length of the amplification product ranges from 50 to 2000 bp, and preferably 60 to 2000 bp. More preferably, the primer set is designed so that the length of longer fragments ranges from 400 to 600 bp, and the length of shorter fragments ranges from 200 to 300 bp. This is because too long fragments can make it difficult to obtain a nucleic acid amplification reaction product, and make it easy to cause extension error during repeated reactions.

Besides the above, in designing primers, it is preferred to design so that the following requirements are satisfied: a GC content is 40 to 60%; a secondary structure is not included inside the primer; a self-complementary sequence is not included in the primer; 3′-ends are not complementary with each other in a combination of primer sets; and a melting temperature for annealing is set to 55 to 65° C.

As described above, primers of (i), (ii), (iii) and (iv) are designed according to the sequence of the target LTR retrotransposon, and primer sets for use in classification and discrimination methods are designed.

Table 2 shows representative examples of primer sets useful for discrimination of LTR-type retrotransposon having a LTR sequence of each family shown in Table 1. Primers shown in Table 2 are representative examples, and other primers satisfying the aforementioned primer designing requirements are also included without limited to the above. In the Table, notations of A, B, C, D, and E in the column of kind indicate the starting point position shown in FIG. 6 for each primer.

| | | | | |
|---|---|---|---|---|
| LTR | Primer | | | SEQ |
| Name | Name | Primer Sequence | Kind | ID NO. |

Table 2-1

| | | | | |
|---|---|---|---|---|
| Jc3S1 | LTR_R2 | TTATTGCCGGGGCCTAACAC | D | 1 |
| Jc3S1 | F_L | TGGAGAATTTGGGTTTGGTC | A | 2 |
| Jc3S1 | F_R | CTCGAGACCTCTCAACGAAC | E | 3 |
| Jc7A | LTR_R | TGAGATTAATTCTTACATAT | D | 4 |
| Jc7A | F_L2 | GAACCAGGATCACGTTCAAC | A | 5 |
| Jc7A | F_R | TCGCCCCACTTACTTTCTTG | E | 6 |
| Jc7B | LTR_L | GAAAATTAAATCCAACATGT | B | 7 |
| Jc7B | LTR_R | GAGATTAATTCTTAACAGAA | D | 8 |
| Jc7B | F_L | CAAAGCACACGAGGATTCAG | A | 9 |
| Jc7B | F_R | CAGGTCCAAATCTCCTCGTG | E | 10 |
| Jc7C | LTR_L2 | ATAGAAAATTAAATCCAACAG | B | 11 |
| Jc7C | LTR_R | GAAATTAATTCTTAACATCT | D | 12 |
| Jc7C | F_L2 | TTGGGTGTTTCAGCCATAAG | A | 13 |
| Jc7C | F_R | AGCCAAAGGTTGGAGAAACC | E | 14 |

Table 2-2

| | | | | |
|---|---|---|---|---|
| Jc8A | LTR_L | TTTCTCTTCATCCGACAAAA | B | 15 |
| Jc8A | LTR_R2 | ATTTTTCCCTCTGTGAAACAG | D | 16 |
| Jc8A | F_L | AGATGCTGATAGGGTTGGTG | A | 17 |
| Jc8A | F_R | CAGCACGGCCTCGTTTATAG | E | 18 |
| Jc8B | LTR_L | TTTCTCTTCATCCGACATGG | B | 19 |
| Jc8B | LTR_R | TTTCCCTCTGTGAAACACCC | D | 20 |
| Jc8B | F_L | GTGGGATCTTGAAGGACCAG | A | 21 |
| Jc8B | F_R | TGTTGAGAAACATGGTCAAGC | E | 22 |
| Jc8C | LTR_L | TTTCTCTTCATCCGACAAAA | B | 23 |
| Jc8C | LTR_R | TTTCTCTCTGTAAAACATCT | D | 24 |
| Jc8C | F_L | TTGCCCAAATTTCACTTCATC | A | 25 |
| Jc8C | F_R2 | CCGAATTTTGAGCCAGCTTG | E | 26 |
| Jc8D | LTR_L | TTTCTCTTCATCCGACATTA | B | 27 |
| Jc8D | LTR_R | TTTCCCTCTGTGAAACAGAT | D | 28 |
| Jc8D | F_L2 | CTTACTGACTTCATTAATTG | A | 29 |
| Jc8D | F_R | CACCCACCCTCTTCTTCATC | E | 30 |
| Jc9A | LTR_L | ACAACTAATCCTTAACATTG | B | 31 |
| Jc9A | LTR_R | CCCACATTAAATCTACAAAC | D | 32 |
| Jc9A | F_L | TGCTTGGATTTAAGCCTTTG | A | 33 |
| Jc9A | F_R2 | CAACCCAACGAGCAAGCTAC | E | 34 |
| Jc9S2 | LTR_L | ACAACTAATCCTTAACAAAC | B | 35 |
| Jc9S2 | LTR_R | CCCACATTAAATCTACAAGG | D | 36 |
| Jc9S2 | F_L | CACTCCTAAAATGCGGCTAAC | A | 37 |
| Jc9S2 | F_R | TTCCACTGCTATTGTTTAATTCAT | E | 38 |

The primers as described above may be appropriately labeled for detection. They may be labeled with labels that are ordinarily used in the art of PCR, for example, dyes, fluorescence, isotopes, and enzymes.

### <Line Classification and Discrimination Method>

By using the primer sets designed as described above to detect the presence or absence of LTR-type retrotransposon, it is possible to classify Jatropha lines that have not been classified yet. In addition, based on the phylogenic tree established by the classification method of the present invention, it is possible to obtain information about to which line the objective Jatropha individual belongs and from which line the individual speciated and evolved.

A method for classifying and discriminating Jatropha lines using the primer sets designed as described above will be described below.

The amplification reaction using the aforementioned primer sets is conducted using DNA prepared from the objective Jatropha individual as a template.

A DNA sample of Jatropha to be subjected to the amplification reaction can be prepared by extracting genome DNA from a Jatropha individual according to the technique commonly used in the field of plant molecular biology. A commercially available DNA extraction reagent (for example, DNeasy Plant Mini Kit) may be used.

For extraction of genome DNA, any tissues of a Jatropha individual (for example, leaf, stem, root, petal, and callus) can be used.

For the objective Jatropha, by detecting the presence or absence of insertion of Jc of specific LTR-type retrotransposon, it is possible to make classification between Line I having Jc retrotransposon and Line II not having Jc retrotransposon in the line chart of FIG. 7. Further, for a different gene locus, the presence or absence of insertion of a different retrotransposon family may be detected. From combination of the presence and absence of insertion of retrotransposon family, it is possible to discriminate between the downstream Line III and Line IV. In detecting the presence or absence of insertion of a different retrotransposon family for a plurality of gene loci, the presence or absence of each retrotransposon may be detected for each gene locus, or may be detected simultaneously by utilizing multiple PCR or the like.

### First Embodiment

The discrimination method of the first embodiment is the case where the primer set of primers (i), (ii) and (iii) is used. In other words, the primer set including a combination of an adjacent forward primer starting at A, an adjacent reverse primer starting at E, and a LTR reverse primer starting at B or a LTR forward primer starting at D in FIG. 6 is used.

In the amplification reaction using this primer set, if the Jatropha individual that is an objective of determination is of Line I into which retrotransposon used as a marker is inserted, amplification products of A-E region (region between A and E) including the target retrotransposon sequence and LTR, and A-B sequence (sequence between A and B) or D-E sequence (sequence between D and E) including a LTR sequence starting from the sequence having complementarity with the LTR forward primer or the LTR reverse primer used as the primer (iii) will be obtained. Usually, the A-E fragment length is too long to achieve stable replication and amplification, and thus a long A-E fragment including the retrotransposon region tends not to be obtained.

On the other hand, if the Jatropha individual that is an objective of determination is of Line II into which retrotransposon used as a marker is not inserted, only an amplification product of a short A-E sequence as shown in FIG. 6 will be obtained.

In such an embodiment, multiple PCR can be conducted when the length of the A-B fragment (or D-E fragment) that is an amplification product of Line I and the length of the A-E fragment that is an amplification product of Line II differ to such a degree that they are easily distinguishable from each other.

When retrotransposon is of a special line consisting only of a LTR sequence, an A-E amplification product is obtained in both of the line having retrotransposon and the line into which retrotransposon is not inserted. However, the obtained amplification products are different in length and kind (sequence) between the A-E amplification product of the line having retrotransposon and the A-E amplification product in which retrotransposon is not inserted. It is thus possible to distinguish these lines from each other according to the difference in kind or fragment length of the amplification product.

### Second Embodiment

The primer set of primers (i), (ii) and (iv) is used. That is, an adjacent forward primer starting at A, an adjacent reverse primer starting at E, and a RTP forward primer starting at C₁ or a RTP reverse primer starting at C₂ shown in FIG. 2 are used.

In the amplification reaction using this primer set, if the Jatropha individual that is an objective of determination is of Line I into which LTR-type retrotransposon Jc used as a marker is inserted, amplification reactions occur in an A-E region including the target retrotransposon sequence and LTR, and in an A-C₂ or C₁-E region including a part of the retrotransposon sequence starting from the sequence having complementarity with the RTP forward primer or the RTP reverse primer used as the primer (iv) and the LTR sequence. Usually, stable replication and amplification reactions are not achieved between A and E because of too long fragment length, and as a result, an amplification product of the long A-E fragment including the retrotransposon region is not obtained, and an amplification product of the A-C₂ region or the C₁-E region is obtained.

On the other hand, when the Jatropha individual that is an objective of determination is of Line II into which the retrotransposon used as a marker is not inserted, only an amplification product of a short A-E sequence as shown in FIG. 6 is obtained.

Therefore, Line I and Line II can be distinguished from each other because the kinds (length, sequence and so on) of the obtained amplification products are different from each other. In other words, whether the Jatropha individual that is an objective of determination belongs to Line I or Line II can be determined according to the kind (length, sequence and so on) of the obtained amplification product.

### Third Embodiment

In the third embodiment, in order that a plurality of retrotransposon families can be detected, corresponding retrotransposon inserted in a different gene locus is detected by using a primer set in which primer pairs for detection of retrotransposon are appropriately combined, and the line is classified and discriminated according to the combination of the inserted retrotransposons.

For example, the presence or absence of insertion of retrotransposon of Jc7 family and the presence or absence of insertion of retrotransposon of Jc8 family are individually detected, and classification is made according to the combination of insertion of these retrotransposons. For example, classification between Line I and Line II can be made according to the presence or absence of insertion of retrotransposon of Jc7 family, and further classification between Line III and Line IV can be made according to the presence or absence of insertion of retrotransposon of Jc8 family.

For detection of individual retrotransposon, the primer pair as used in the first embodiment or the second embodiment can be selected.

In detection of a plurality of retrotransposons, the retrotransposons may be detected sequentially by using respective primer pairs for detecting the retrotransposons, or may be detected by simultaneously obtaining amplification products by utilizing multiple PCR or the like using a combination of primer pairs for detecting the plurality of retrotransposons.

### Fourth Embodiment

In detection of retrotransposon in the fourth embodiment, (i) adjacent forward primer, (ii) adjacent reverse primer, (iii) LTR forward or reverse primer, and (iv) RTP forward or reverse primer are involved.

This embodiment is useful when it is desired to discriminate lines in consideration of difference in LTR polymorphs or in polymorphs of the adjacent region.

In the amplification reaction using this primer set, if the Jatropha individual that is an objective of determination is of Line I into which retrotransposon used as a marker is inserted, both the amplification products obtained in the first embodiment and the second embodiment, namely, the amplification product of A-B sequence or D-E sequence including the LTR sequence, and the amplification product of A-C₂ sequence or C₁-E sequence will be obtained. Here, for example, when the sequence of LTR or the adjacent region does not coincide with the sequence used as the primer due to mutation in Line IV, the A-C₂ or C₁-E amplification product is obtained, but the A-B or D-E amplification product is not obtained. On the other hand, in the case of Line III having a LTR sequence that is coincident with the LTR used as a primer, an amplification product of the A-B sequence or D-E sequence, and an amplification product of A-C₂ or C₁-E will be obtained.

On the other hand, if the Jatropha individual that is an objective of determination is of Line II into which retrotransposon used as a marker is not inserted, only an amplification product of a short A-E sequence will be obtained.

As described above, it is possible to determine whether or not the Jatropha individual that is an objective of determination is of Line II not having retrotransposon used as a marker, and further to determine to which line (for example, Line III or Line IV shown in FIG. 7) classified by LTR polymorphism the individual belongs when it is of Line I into which retrotransposon is inserted.

The nucleic acid amplification reaction in the above first to fourth embodiments may be conducted by known nucleic acid amplification means that is appropriately selected. Concrete examples include, but not limited to, a PCR method, an ICAN (Isothermal and Chimeric primer-initiated Amplification of Nucleic acids) method, a UCAN method, a LAMP (Loop-Mediated Isothermal Amplification) method, and a primer extension method. Among these, the PCR method is preferred. For detecting the presence or absence of a plurality of retrotransposon families, multiple PCR that conducts an amplification reaction using a combination of a plurality of primers followed by detection may be employed.

The amplification reaction is conducted by using DNA prepared from an objective Jatropha individual as a template and a primer set specific to the line to be discriminated. By using a set of primer pairs for detecting a plurality of retrotransposons, it is possible to make classification and discrimination regarding to which line unclassified Jatropha belongs.

As a method for checking the presence or absence of an amplification product, for example, electrophoresis such as agarose gel electrophoresis, and acrylamide gel electrophoresis can be recited. Electrophoresis can detect the presence or absence and difference in length of amplification products. Since the length of amplification products detected in this context indicates the presence or absence of the target sequence (a part of a LTR sequence or retrotransposon sequence), such strictness as detecting difference of several bases is not required. Therefore, it can be detected even with simple electrophoresis such as agarose gel electrophoresis.

For example, when the primer set of the first embodiment is used, an amplification product of A-B or D-E is obtained in a line having predetermined LTR-type retrotransposon. On the other hand, only an amplification product of A-E is obtained in a line not having predetermined LTR-type retrotransposon. The amplification product of A-B or D-E is typically designed to have 200 to 300 bp, and hence the obtained fragment has 200 to 300 bp depending on the kind of a primer. On the other hand, the A-E amplification product obtained from the variety not having LTR-type retrotransposon typically has about 400 to 600 bp. Thus, since the compared fragment lengths differ nearly twice, they can be easily distinguished from each other because different bands are obtained as a result of electrophoresis even with multiple PCR. In other words, it is possible to distinguish the line having a predetermined LTR-type retrotransposon from the line not having the same according to the obtained fragment lengths.

For determination of LTR polymorphism, it is preferred to detect polymorphs, for example, by conducting the amplification reaction in a similar manner as described above and then determining the sequence of the amplification product. When the polymorphism is known, the kind of the inserted retrotransposon family may be determined by detecting the amplification product that hybridizes with a probe, by using a probe array prepared by immobilizing a probe capable of hybridizing with the known LTR sequence to a support.

In this manner, it is possible to classify the unclassified lines of Jatropha. Then, it is possible to determine to which line each Jatropha individual belongs on the basis of the relation classification with various LTR family members. In addition, by examining the sequence of the amplification product, it is possible to discriminate lines of closely-related species of the same line.

### Examples

Modes for carrying out the present invention will be described by way of Examples. The following Examples are not intended to limit the scope of the present invention.

### <Samples of Jatropha>

As samples for classification and discrimination, leaves of Jatropha of the following lines were collected.

Those of Asia and Africa line: from Uganda, from Cape verde, from Madagascar, from Tanzania, from China, from Thai, from Indonesia (Indonesia IS), and from Parawan (Philippines).

Those of Mexico and Guatemala line: from Mexico (Mexico 2b), and from Guatemala (Guatemala 1 and 2).

### <Amplification by PCR and Classification and Discrimination of Lines>

From leaves of Jatropha of various lines, DNA was extracted by a CTAB (Cetyl trimethyl ammonium bromide) method, and a sample for discrimination was prepared.

For each sample for discrimination, PCR was conducted in the following condition by using a primer group (LTR_L, LTR_R, F_L, F_R) for detecting LTR of Jc7B shown in Table 2 to obtain a PCR product. A PCR reaction was conducted for each sample by using a primer pair 1 (F_L and LTR_L), a primer pair 2 (LTR_R and FR), or a primer pair 3 (F_L and F_R).

PCR Condition:

After keeping at 94° C. for 2 minutes, a cycle consisting of 94° C. for 15 seconds, 55° C. for 45 seconds, and 72° C. for 2 minutes was repeated 35 times, followed by an extension reaction at 72° C. for 10 minutes.

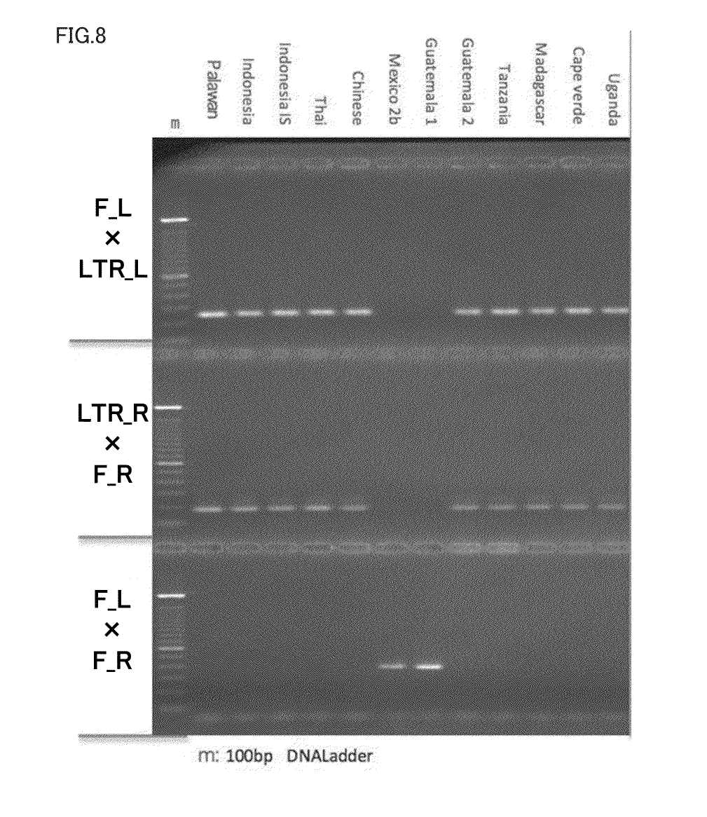

The obtained PCR product was subjected to electrophoresis in 2% agarose gel prepared in a TAE buffer at 100 V for 40 minutes. As an index for the length of the amplification product, a 100 bp DNA Lader marker was used. After the end of electrophoresis, the gel was removed from the gel plate, and stained with ethidium bromide, and then a DNA band was observed under UV radiation. The gel image is shown in FIG. 8.

As shown in FIG. 8, in Jatropha of Uganda, Cape verde, Madagascar, Tanzania, Chinese, Thai, Indonesia IS, and Parawan belonging to Asia and Africa line, an amplification product was obtained and a corresponding band was observed when the primer pair 1 (A-B amplification product) or the primer pair 2 (D-E amplification product) for amplifying LTR and the adjacent region was used. On the other hand, in Jatropha of Mexico 2b and Guatemala 1 belonging to Mexico and Guatemala line, an amplification product was not obtained and a corresponding band was not observed when the primer pair 1 or the primer pair 2 was used, however, an amplification product was obtained (short A-E amplification product) and a band was observed at about 385 bp when the primer pair 3 was used. Therefore, existence of retrotransposon of Jc7 is considered as a common characteristic in Asia and Africa line.

As to Guatemala 2, an amplification product was obtained and a corresponding band was observed when the primer pair 1 or the primer pair 2 was used although it is of Mexico and Guatemala line. This result suggests that Guatemala 2 is of a line closer to Asia and Africa line than to Mexico 2b and Guatemala 1. In other words, it is supposed that a common ancestor of Mexico 2b and Guatemala 1, and a common ancestor of Guatemala 2 and Asia and Africa line first branched, and then Guatemala 2 and Asia and Africa line speciated.

It is found that further line classification in Mexico and Guatemala line is possible by focusing on retrotransposon of Jc7.

The discrimination method of Jatropha of the present invention is useful as a method for classifying and discriminating various lines of Jatropha, since classification and discrimination can be made for remote lines of Jatropha in consideration of the course of evolution, and for close lines based on LTR polymorphism, if desired.

What is claimed is:

1. A method for classifying and discriminating Jatropha lines using a LTR (Long Terminal Repeat)-type retrotransposon marker, comprising the steps of:
conducting a nucleic acid amplification reaction using a primer set including primers of the following (i) and (ii), and a primer of the following (iii) or (iv), wherein DNA prepared from Jatropha that is an objective of determination, is used as a template:
(i) a forward primer of 15 to 60 bases having complementarity with a base sequence of 5′-side adjacent region of left LTR (hereinafter, referred to as “adjacent forward primer”),
(ii) a reverse primer of 15 to 60 bases having complementarity with a base sequence of 3′-side adjacent region of right LTR (hereinafter, referred to as “adjacent reverse primer”),
(iii) a forward primer of 15 to 60 bases having complementarity with 3′-terminal sequence of said right LTR sequence and its 3′-side adjacent sequence, or a reverse primer of 15 to 60 bases having complementarity with 5′-terminal sequence of said left LTR sequence and its 5′-side adjacent sequence (hereinafter, referred to as “LTR primer”), and
(iv) a forward or reverse primer of 15 to 60 bases having complementarity with at least a part of a retrotransposon sequence existing between said left and right LTR sequences (hereafter, referred to as “RTP forward primer” or “RTP reverse primer”); and
determining the presence or absence of insertion of LTR-type retrotransposon including the retrotransposon sequence used in (iii) or (iv), on the basis of the presence or absence and length of an amplification product obtained in said amplification reaction.

2. The method for classifying and discriminating Jatropha lines according to claim 1, wherein the above (i), (ii), and the LTR forward primer and the LTR reverse primer of (iii) are used as said primer set.

3. The method for classifying and discriminating Jatropha lines according to claim 1, wherein said retrotransposon is at least one selected from the group consisting of retrotransposons represented by Jc3 (SEQ ID NO: 49), Jc7 (SEQ ID NO: 50), Jc8 (SEQ ID NO: 51), and Jc9 (SEQ ID NO: 52), and sequences having a homology of greater than or equal to 85% with said respective sequences.

4. The method for classifying and discriminating Jatropha lines according to claim 1, wherein said LTR has a base sequence having a homology of greater than or equal to 85% with a sequence represented by one selected from SEQ ID NOs: 39 to 41.

5. The method for classifying and discriminating Jatropha lines according to claim 1, wherein the presence or absence of LTR-type retrotransposon of Jc3S is detected by using a primer set consisting of primers represented by SEQ ID NOs: 1, 2 and 3, or primers having a sequence identity of greater than or equal to 80% in 15 bases on the 3′-end side of said respective primers.

6. The method for classifying and discriminating Jatropha lines according to claim 1, wherein the presence or absence of LTR-type retrotransposon of Jc9S is detected by using a primer set consisting of primers represented by SEQ ID NOs: 35, 36, 37 and 38, or primers having a sequence identity of greater than or equal to 80% in 15 bases on the 3′-end side of said respective primers.

7. The method for classifying and discriminating Jatropha lines according to claim 1, wherein as said primer set, a plurality of primer sets are used for determining the presence or absence of insertion of a plurality of retrotransposons, and the method further comprising the step of classifying and discriminating Jatropha lines according to the presence or absence of insertion of each retrotransposon.

8. The method for classifying and discriminating Jatropha lines according to claim 1, wherein an amplification product of a line including retrotransposon has a fragment length of 50 to 2000 bp.

9. The method for classifying and discriminating Jatropha lines according to claim 1, wherein said nucleic acid amplification reaction is a polymerase chain reaction (PCR).

10. The method for classifying and discriminating Jatropha lines according to claim 1, wherein the length of said amplification product is distinguished by agarose gel electrophoresis or polyacrylamide gel electrophoresis.

11. The method for classifying and discriminating Jatropha lines according to claim 1, wherein said primer sets based on the following LTR sequences are as follows:
(1) when said LTR is the sequence of SEQ ID NO: 39 or has a homology of greater than or equal to 85% with the sequence, (a) a primer set of at least one primer selected from the group consisting of primers of SEQ ID NOs: 1, 2 and 3;
(2) when said LTR is the sequence of SEQ ID NO: 40 or has a homology of greater than or equal to 85% with the sequence, (b) a primer set of at least two primers selected from the group consisting of primers of SEQ ID NOs: 4, 5 and 6;
(3) when said LTR is the sequence of SEQ ID NO: 41 or has a homology of greater than or equal to 85% with the sequence, (c) a primer set of at least two primers selected from the group consisting of primers of SEQ ID NOs: 7, 8, 9 and 10;
(4) when said LTR is the sequence of SEQ ID NO: 42 or has a homology of greater than or equal to 85% with the sequence, (d) a primer set of at least two primers selected from the group consisting of primers of SEQ ID NOs: 11, 12, 13 and 14,
(5) when said LTR is the sequence of SEQ ID NO: 43 or has a homology of greater than or equal to 85% with the sequence, (e) a primer set of at least two primers selected from the group consisting of primers of SEQ ID NOs: 15, 16, 17 and 18;
(6) when said LTR is the sequence of SEQ ID NO: 44 or has a homology of greater than or equal to 85% with the sequence, (f) a primer set of at least two primers selected from the group consisting of primers of SEQ ID NOs: 19, 20, 21 and 22;
(7) when said LTR is the sequence of SEQ ID NO: 45 or has a homology of greater than or equal to 85% with the sequence, (g) a primer set of at least two primers selected from the group consisting of primers of SEQ ID NOs: 23, 24, 25 and 26;
(8) when said LTR is the sequence of SEQ ID NO: 46 or has a homology of greater than or equal to 85% with the sequence, (h) a primer set of at least two primers selected from the group consisting of primers of SEQ ID NOs: 27, 28, 29 and 30;
(9) when said LTR is the sequence of SEQ ID NO: 47 or has a homology of greater than or equal to 85% with the sequence, (i) a primer set of at least two primers selected from the group consisting of primers of SEQ ID NOs: 31, 32, 33 and 34; and
(10) when said LTR is the sequence of SEQ ID NO: 48 or has a homology of greater than or equal to 85% with the sequence, (j) a primer set of at least one primer selected from the group consisting of primers of SEQ ID NOs: 35, 36, 37 and 38.

12. A kit for classifying and discriminating Jatropha lines comprising at least one primer set selected from the following primer sets:
(a) a primer set of at least one primer selected from the group consisting of primers of SEQ ID NOs: 1, 2 and 3, and primers having a sequence identity of greater than or equal to 80% in 15 bases on the 3′-end side of said respective primers;
(b) a primer set of at least two primers selected from the group consisting of primers of SEQ ID NOs: 4, 5 and 6, and primers having a sequence identity of greater than or equal to 80% in 15 bases on the 3′-end side of said respective primers;
(c) a primer set of at least two primers selected from the group consisting of primers of SEQ ID NOs: 7, 8, 9 and 10, and primers having a sequence identity of greater than or equal to 80% in 15 bases on the 3′-end side of said respective primers;
(d) a primer set of at least two primers selected from the group consisting of primers of SEQ ID NOs: 11, 12, 13 and 14, and primers having a sequence identity of greater than or equal to 80% in 15 bases on the 3′-end side of said respective primers;
(e) a primer set of at least two primers selected from the group consisting of primers of SEQ ID NOs: 15, 16, 17 and 18, and primers having a sequence identity of greater than or equal to 80% in 15 bases on the 3′-end side of said respective primers;
(f) a primer set of at least two primers selected from the group consisting of primers of SEQ ID NOs: 19, 20, 21 and 22, and primers having a sequence identity of greater than or equal to 80% in 15 bases on the 3′-end side of said respective primers;
(g) a primer set of at least two primers selected from the group consisting of primers of SEQ ID NOs: 23, 24, 25 and 26, and primers having a sequence identity of greater than or equal to 80% in 15 bases on the 3′-end side of said respective primers;
(h) a primer set of at least two primers selected from the group consisting of primers of SEQ ID NOs: 27, 28, 29 and 30, and primers having a sequence identity of greater than or equal to 80% in 15 bases on the 3′-end side of said respective primers;
(i) a primer set of at least two primers selected from the group consisting of primers of SEQ ID NOs: 31, 32, 33 and 34, and primers having a sequence identity of greater than or equal to 80% in 15 bases on the 3′-end side of said respective primers; and
(j) a primer set of at least one primer selected from the group consisting of primers of SEQ ID NOs: 35, 36, 37 and 38, and primers having a sequence identity of greater than or equal to 80% in 15 bases on the 3′-end side of said respective primers.

13. The kit for classifying and discriminating Jatropha lines according to claim 12, comprising all of the following primer sets:
(a) a primer set of at least one primer selected from the group consisting of primers of SEQ ID NOs: 1, 2 and 3, and primers having a sequence identity of greater than or equal to 80% in 15 bases on the 3′-end side of said respective primers;
(b) a primer set of at least two primers selected from the group consisting of primers of SEQ ID NOs: 4, 5 and 6, and primers having a sequence identity of greater than or equal to 80% in 15 bases on the 3′-end side of said respective primers;
(c) a primer set of at least two primers selected from the group consisting of primers of SEQ ID NOs: 7, 8, 9 and 10, and primers having a sequence identity of greater than or equal to 80% in 15 bases on the 3′-end side of said respective primers;
(d) a primer set of at least two primers selected from the group consisting of primers of SEQ ID NOs: 11, 12, 13 and 14, and primers having a sequence identity of greater than or equal to 80% in 15 bases on the 3′-end side of said respective primers;
(e) a primer set of at least two primers selected from the group consisting of primers of SEQ ID NOs: 15, 16, 17 and 18, and primers having a sequence identity of greater than or equal to 80% in 15 bases on the 3′-end side of said respective primers;
(f) a primer set of at least two primers selected from the group consisting of primers of SEQ ID NOs: 19, 20, 21 and 22, and primers having a sequence identity of greater than or equal to 80% in 15 bases on the 3′-end side of said respective primers;
(g) a primer set of at least two primers selected from the group consisting of primers of SEQ ID NOs: 23, 24, 25 and 26, and primers having a sequence identity of greater than or equal to 80% in 15 bases on the 3′-end side of said respective primers;
(h) a primer set of at least two primers selected from the group consisting of primers of SEQ ID NOs: 27, 28, 29 and 30, and primers having a sequence identity of greater than or equal to 80% in 15 bases on the 3′-end side of said respective primers;
(i) a primer set of at least two primers selected from the group consisting of primers of SEQ ID NOs: 31, 32, 33 and 34, and primers having a sequence identity of greater than or equal to 80% in 15 bases on the 3′-end side of said respective primers; and
(j) a primer set of at least one primer selected from the group consisting of primers of SEQ ID NOs: 35, 36, 37 and 38, and primers having a sequence identity of greater than or equal to 80% in 15 bases on the 3′-end side of said respective primers.


SEQUENCE LISTING (52 sequences)

SEQ ID NO: 1 - DNA, 20 bp, Artificial Sequence
  Description of Artificial Sequence Synthetic primer
ttattgccggggcctaacac

SEQ ID NO: 2 - DNA, 20 bp, Artificial Sequence
  Description of Artificial Sequence Synthetic primer
tggagaatttgggtttggtc

SEQ ID NO: 3 - DNA, 20 bp, Artificial Sequence
  Description of Artificial Sequence Synthetic primer
ctcgagacctctcaacgaac

SEQ ID NO: 4 - DNA, 20 bp, Artificial Sequence
  Description of Artificial Sequence Synthetic primer
tgagattaattcttacatat

SEQ ID NO: 5 - DNA, 20 bp, Artificial Sequence
  Description of Artificial Sequence Synthetic primer
gaaccaggatcacgttcaac

SEQ ID NO: 6 - DNA, 20 bp, Artificial Sequence
  Description of Artificial Sequence Synthetic primer
tcgccccacttactttcttg

SEQ ID NO: 7 - DNA, 20 bp, Artificial Sequence
  Description of Artificial Sequence Synthetic primer
gaaaattaaatccaacatgt

SEQ ID NO: 8 - DNA, 20 bp, Artificial Sequence
  Description of Artificial Sequence Synthetic primer
gagattaattcttaacagaa

SEQ ID NO: 9 - DNA, 20 bp, Artificial Sequence
  Description of Artificial Sequence Synthetic primer
caaagcacacgaggattcag

SEQ ID NO: 10 - DNA, 20 bp, Artificial Sequence
  Description of Artificial Sequence Synthetic primer
caggtccaaatctcctcgtg

SEQ ID NO: 11 - DNA, 21 bp, Artificial Sequence
  Description of Artificial Sequence Synthetic primer
atagaaaattaaatccaacag

SEQ ID NO: 12 - DNA, 20 bp, Artificial Sequence
  Description of Artificial Sequence Synthetic primer
gaaattaattcttaacatct

SEQ ID NO: 13 - DNA, 20 bp, Artificial Sequence
  Description of Artificial Sequence Synthetic primer
ttgggtgtttcagccataag

SEQ ID NO: 14 - DNA, 20 bp, Artificial Sequence
  Description of Artificial Sequence Synthetic primer
agccaaaggttggagaaacc

SEQ ID NO: 15 - DNA, 20 bp, Artificial Sequence
  Description of Artificial Sequence Synthetic primer
tttctcttcatccgacaaaa

SEQ ID NO: 16 - DNA, 21 bp, Artificial Sequence
  Description of Artificial Sequence Synthetic primer
atttttccctctgtgaaacag

SEQ ID NO: 17 - DNA, 20 bp, Artificial Sequence
  Description of Artificial Sequence Synthetic primer
agatgctgatagggttggtg

SEQ ID NO: 18 - DNA, 20 bp, Artificial Sequence
  Description of Artificial Sequence Synthetic primer
cagcacggcctcgtttatag

SEQ ID NO: 19 - DNA, 20 bp, Artificial Sequence
  Description of Artificial Sequence Synthetic primer
tttctcttcatccgacatgg

SEQ ID NO: 20 - DNA, 20 bp, Artificial Sequence
  Description of Artificial Sequence Synthetic primer
tttccctctgtgaaacaccc

SEQ ID NO: 21 - DNA, 20 bp, Artificial Sequence
  Description of Artificial Sequence Synthetic primer
gtgggatcttgaaggaccag

SEQ ID NO: 22 - DNA, 21 bp, Artificial Sequence
  Description of Artificial Sequence Synthetic primer
tgttgagaaacatggtcaagc

SEQ ID NO: 23 - DNA, 20 bp, Artificial Sequence
  Description of Artificial Sequence Synthetic primer
tttctcttcatccgacaaaa

SEQ ID NO: 24 - DNA, 20 bp, Artificial Sequence
  Description of Artificial Sequence Synthetic primer
tttctctctgtaaaacatct

SEQ ID NO: 25 - DNA, 21 bp, Artificial Sequence
  Description of Artificial Sequence Synthetic primer
ttgcccaaatttcacttcatc

SEQ ID NO: 26 - DNA, 20 bp, Artificial Sequence
  Description of Artificial Sequence Synthetic primer
ccgaattttgagccagcttg

SEQ ID NO: 27 - DNA, 20 bp, Artificial Sequence
  Description of Artificial Sequence Synthetic primer
tttctcttcatccgacatta

SEQ ID NO: 28 - DNA, 20 bp, Artificial Sequence
  Description of Artificial Sequence Synthetic primer
tttccctctgtgaaacagat

SEQ ID NO: 29 - DNA, 20 bp, Artificial Sequence
  Description of Artificial Sequence Synthetic primer
cttactgacttcattaattg

SEQ ID NO: 30 - DNA, 20 bp, Artificial Sequence
  Description of Artificial Sequence Synthetic primer
cacccaccctcttcttcatc

SEQ ID NO: 31 - DNA, 20 bp, Artificial Sequence
  Description of Artificial Sequence Synthetic primer
acaactaatccttaacattg

SEQ ID NO: 32 - DNA, 20 bp, Artificial Sequence
  Description of Artificial Sequence Synthetic primer
cccacattaaatctacaaac

SEQ ID NO: 33 - DNA, 20 bp, Artificial Sequence
  Description of Artificial Sequence Synthetic primer
tgcttggatttaagcctttg

SEQ ID NO: 34 - DNA, 20 bp, Artificial Sequence
  Description of Artificial Sequence Synthetic primer
caacccaacgagcaagctac

SEQ ID NO: 35 - DNA, 20 bp, Artificial Sequence
  Description of Artificial Sequence Synthetic primer
acaactaatccttaacaaac

SEQ ID NO: 36 - DNA, 20 bp, Artificial Sequence
  Description of Artificial Sequence Synthetic primer
cccacattaaatctacaagg

SEQ ID NO: 37 - DNA, 21 bp, Artificial Sequence
  Description of Artificial Sequence Synthetic primer
cactcctaaaatgcggctaac

SEQ ID NO: 38 - DNA, 24 bp, Artificial Sequence
  Description of Artificial Sequence Synthetic primer
ttccactgctattgtttaattcat

SEQ ID NO: 39 - DNA, 536 bp, Jatropha sp.
tgttgtgcggtgcccactcagtgggacccacactttgttgaaaataaataatggtaggtaatcaaccacacgtagttgaaaagaaaaaaaaaaaaaaaagaagaaaattggagaaagtctttggtaggtgatctgctggtaggtggatgcgttaatcacgcatccatctctcctctaatcaagcttccacatctcctataaataggagatggtgctagagcttcaatgcaccaacacgcagagcgacacagagtaagattgagagagttttaagttttagaattgggttctaaaacttgggtagttaaaatagtgaagataatttagggtgtttttgggaaacccgtgtgagtgacactatttgtatattactctctttgtacgcctactatttttaaatagtggaagaattattcggtttttgtcccgtggacgtagccctaaacagttaagggtgaaccacgtaaatatctgtgtgtcatatttatttttctgctgtgttattattctcgtaattttattgccggggcctaaca

SEQ ID NO: 40 - DNA, 446 bp, Jatropha sp.
tgttggatttaattttctattgtgggcttaatatgtaaaattaaatatgtacagcagtctaattgtaaagtcaatttcaaatgtggtcttgattgtgaataattgactattgaggtatcaactaattatagatattaagtgtagagaccattatataattcctatttcgttaaaggaccgaaataggaattgtactaaccctacaggtatataaaggtcgtggtccccagaggagttgaatacgattcagttttacgttcctttcgtattgcttccgctaaatcgattcttcaattctctggaaggctgcaaacctaattctatacatcaagatcgacgaacaacgatgtcaggtacgcaaccttttcttattcaatctattttgttgaatctagtggtttgcacgatctaggttattgtggaattatttgagattaattcttaca

SEQ ID NO: 41 - DNA, 447 bp, Jatropha sp.
tgttggatttaattttctattgtgggcttaatatgtaaaattaaatatgtacagcagtccaattgtaaagtcaatttcaaatgtggtcttgattgtgaataattgactattgaggtatcaactaattatagatattaagtgtagagaccattatataattcctatttcgttaaaggaccgaaataggaattgtactaaccctacaggtatataaaggtcgtggtccccagaggagttgaatacgattcagttttacgttcctttcgtactgcttccgcaaaatcgattcttcaattctctggaaggctgcaaacctaattctacacatcaagatcgacgaacaacgatgtcaggtacgcaaccttttcttattcaatctattttgttgaatctagtgatttgcacgatctaggttattgtggaattatttgagattaattcttaaca

SEQ ID NO: 42 - DNA, 447 bp, Jatropha sp.
tgttggatttaattttctattgtgggcttaatatgtaaaattaaatatgtacagcagtctaattgtaaagtcaatttcaaatgtggtcttgattgtgaataattgactattgaggtatcaactaattatagatattaagtgtagagaccattatataattcctatttcgttaaaggaccgaaataggaattgtactaaccctacaggtatataaaggtcgtggtccccagaggagttgaatacgattcagttttacgttcctttcgtattgcttccgctaaatcgattcttcaattctctggaaggctgcaaacctaattctatacatcaagatcgacgaacaacgatgtcaggtacgcaaccttttcttattcaatctattttgttgaatctagtggtttgcacgatctaggttattattgaattatttgaaattaattcttaaca

SEQ ID NO: 43 - DNA, 210 bp, Jatropha sp.
tgtcggatgaagagaaaaagtagggatcagaatcatagttcatcatttccctgtccactaacctgtacacacagtaaaagtaaaaatcatgttaggtggaactttaggagtaattagagccgtaattagtggatagttagaagcagtttgtatattaaaacaatgtaatctttacagaaaaagaagaacaatttttccctctgtgaaaca

SEQ ID NO: 44 - DNA, 209 bp, Jatropha sp.
tgtcggatgaagagaaaaagtagggatcagaatcatagttcatcatttccctgtccactaaactgtacacacagtaaaagtaaaaatcatgttaggtggaactttaggagtaattagagccgtaattagtggatagttagaagcagtttgtatattaaaacaatgtaatctttacagaaaataataacaatttttccctctgtgaaaca

SEQ ID NO: 45 - DNA, 212 bp, Jatropha sp.
tgtcggatgaagagaaaaagtagggatcagaatcatagttcatcatttctctgtccactaacctgtacacacagtaaaagtaaaagtcaatgttaggtggaactttaggagcaattagagccgtaattagtggatagttagaagcagtttgtatattaaaaacaatgtaatctttacagaagaaatataacaatttttctctctgtaaaaca

SEQ ID NO: 46 - DNA, 210 bp, Jatropha sp.
tgtcggatgaagagaaaaagtagggatcagaatcatagttcatcatttccctgtcaactaacctgtacacacagtaaaagtaaaaatcatgttaggtggaactttaggagtaattagagccgtaattagtggatagttagaagcagtttgtatattaaaacaatgtaatctttacagaaaaaaaagaacaatttttccctctgtgaaaca

SEQ ID NO: 47 - DNA, 161 bp, Jatropha sp.
tgttaaggattagttgtaaatagaactctattaggagttgtaaatagaactctattaggagttagattcttaataggtttatagtatcttgtatgtgtatatatatgtgtccctattgatgaatcaaatacagaaaaatattttcccacattaaatctaca

SEQ ID NO: 48 - DNA, 161 bp, Jatropha sp.
tgttaaggattagttgtaaatagaactctattaggagttgtaaatagaactctattaggagttagattctaaataggtttatagtatcttgtatgtgtatatatatgtgtccctattgatgaatcaaatatagaaaaatattttcccacattaaatctaca

SEQ ID NO: 49 - DNA, 5387 bp, Jatropha sp.
tgttgtgcggtgcccactcagtgggacccacactttgttgaaaatagaagatggtaggtgatcaaccacatgtagtttaaaaaaaaaaaaaaaaaaaagaaaaagtctttggtaggtgatccgttggtaggtggacgcgttaatcacgcatccacctctcctaatcaagcttccacctctcctataaataggagatggtgctagagcttcaatacaccaacaagcagagcaacgcagagagagattgagagagttttgagttttagaattgggttctaaaacttgggtagttaaaatagtgaagataatttagggtgtttttgggaaacacttttgagtgccactattttgtattactctctttgtaagcctactatttttaatagtggaagaaatattcggtttttgtcccgtggacgtagccctaaacagttaaagggtgaaccacgtaaatatctgtgtgtcatatttatatttttccgctgtgttattattctcgtaattttattgccgggccctaacaagtggtatcagagccgaaggtttaccggatccgtggatatattttgaaaataaatataccacgacgacgagaaaattacgccgagtccattggtgcagtccgttcggaaagacgttgcagaatcgacttagtgatgaatagtaaaatagtaactcaaagttgatggtgaacagtactattggcgttactgttgctgagtccgtttcaagtaagcccagcactattcacgcatagtactattcacgtatacagttactattcacgcatacaggtactgtttacgtatactgttactattcagtcatacggtcactattcacgtatactactattcacgtatacggtttttgttggtgataatattctgcacaagtcaaaaatggacgacagaacaacaggcacaatggttagactcaactcatcaaactatgcagtatggaagatattaatggaagatcttctatgctgcaaagatctttttgaaacgattgagttgaaaggggtcaggccaacttcaataaatgagaccgattggaagtcgattaacagaaaagcagtcgcaaccatcaggcagtctgttgaaatgagcgtcataaatcatatcgctcatgagaaagatgcctattcaatttggaagaagttggaggacatgtacgagcggaagaatgcccagaacaaagcttttttaattaaaaagcttgtaaatctaaagtacagagatggcgaaagtgcttcagatcacttgggcaaatttcaagaaatattgaaccaactgacgacgtcaaaaatagtgtttgacgaccaggtgcaggcactattcttattgagttccttgccggacagttgggagacgctgactgttacgctcagtaatgtgtcagaggggaatctgaccttaaccacagttaaggaaagcattctgaatgaagaaaccagacgtaaggagaatggtacgagtagcaactcagatgcactcgttacagttacagaaaggagggggagaagcaaaaccagatacccccaaaatccagacaaacgtgacaattcacggagaagatccaagtcaagaaaaggtgtcacttgctatcattgtggcaagcccggtcacatccaaaggcactgcagaaagtggaagaaggaacgagccaaagaaaatgatgaggaaaaagacgatacggaggtagcttcagtagctattgatgctgacgcacttggtgatggtagtgcgctgatttctcatgctggtctcagtgagtgttccactagtcaacaatctctttggattattgactctgcatgttcttatcatatttcttcaaagaaggaatatttctcatcacttaccctcggagattacggccatgtcaagactttgaatggcacatatgtgaaaaattgcggcattggaacagtatgcatcaaaaccaatgctggttatgatctctggttggaagatgtgagatacactcctacggcaaacttcaatctgatctcagttcattgtcttgatgaattgggctatgcaaatctgtttgtcaacagaacgtggaagctttccaaaggtaacttggtgttcggtagaggaagaaagttcaacaccctctactacacccaggccaaagtttgcacaggagtggtgaatgcagttgaagattcatctgctgagttgtggcacaaacgacttggtcatttgagtgagaaaggaatggagactctcactaagaagaaaatgctatctttgaaaggtttgcctctgaaaatatgcactgattgtttgtctggtaaacaacatagagtttcctttcaaagatccccttctccatctagaaaacctggtattctagaattggtacacactgatgtttgctcaatgggtgacaaatccttgagtggttgctcatattatgttagctttattgatgaccattctagaaaggtgtgggctttaccaataaagtcaaaagaccaggtgctggatatgtttaaagtctttcatgtcagtgttgaaagacaaacgggaagaaagctaaaatgtgttagagctgataatggtggtgagtacagaggaccatttgagaagtattgctcagaacacggcatcagacttcagaagacagttcccaaaactccacaacagaatggagttgcagagagaatgaacaggaccattgttgaaagggttagatgtatgctctctcatgctaaacttcctaaaaccttttgggctgaagcaatgaatactgcagtttacttgatcaacctttcaccttcaacctccctcaacggagatgtaccagaaaaggtgtggtcagggaaaaatatttcttacgatcatcttagaatatttggctgcagggcttttgttcatattccaaaagatgaaagatccaagcttgattcgaagtcaaaacagtgtatcttcttgggttatggaaatgatgattttgggtacagattatgggatccagttgaaaagaagctcatcagaagcagagatgtcgtattcctggaagaccaaaccattgaagactttgagaaaaatgaaaagccaaagtctatcaatgagtttcctgttagtcttgatgcagaacctcgtgtagaaccacaaaatgatgaagaagatgatggtgaacctgttcagaatgatgctgaaccagctccaatagaagaactgcctcaattgagaagatctgcgagaggcaggataccttcccagagatattctcctaatgagtatgacttgttgactcaagggggagaaccagagaactaccaggaggcaatggaagattgtaaaaagaatgagtggttgaaagccatgcaagatgagatggagtccttgcatgagaaccagacttatgagttggtgaaactgcctcaaggtaagaaggcactgaagaacaaatgggtgttcaggttgaagactgaacagaacaactcacagccaaggtacaaggcaagactggttgtgaagggattcggtcagaagaaaggagttgactttgaagaaattttctcacccgtagtcaagatgtcttctatcagagttgtgctcggtttagcagctagcttgaatttggagatcgagcaacttgatgtaaagaccgctttcctccatggtgacttagaggaggaaatttatatggagcagccagaagggttcagagtcaaaggcaaagaacacttggtgtgtaaattgcagaagagcttgtatggcctcaaacaagctcccagacagtggtacaaaaagtttgactctttcatggtaaatcacgggtatgagaaaactacttctgatcattgtgtatttgtgaaaaagttcgtagaaggcgactttattattctcttgttatatgtggatgatatgcttattgtgggccaagatgctaagaaaattgagagcttgaaaaaggagcttggaaaatcctttgccatgaaggatttgggatcggcaaaacagattctcggaatgaagatctccagagacaggaaaaagagaaagttgtggctgtctcaagagaagtacatcgagaaagttctcagcagattcaacatgagcaatgcgaaaccagtttgtactccacttgcaggtcatttcaaattaagtctcagtcaatgtccttcaagtgagaaagaaaaagaagaactgaagaagattccttatgcatccgccgttggcagcttgatgtacgccatggtatgcacaagaccggatattgctcatgctgttggtgttgttagccgatttctttcaaatccaggtaaagaacactggaatgcagtgaagtggattctcaggtatcttaagggcacttccaaaatctgcttatgttttggtgataatagacctgtgttagatggttacacagatgcagatatgggtggtgatgttgattcgcgcaaatcaacttctggatatctgatgacctttgcaggagcagcagtttcttggcaatcaaaattgcaaaaatgcgttgcactctcatccactgaggcggaatatatcgccattacagaagctagcaaagaactcttgtggatgaagaaattcctacaagaattgggtgtagaacaagagaagtacattctgctatgtgatagtcaaagcgcgatacatctcagcaagaattcgacctttcattcaaggtcaaagcacattgatgtgaggtatcattggatacgtgatgcgctggagatgaagttatttcagattgaaaagattcacactgataataatggcgctgatatgatgactaaagctttacctacggagaaacttgtcacttgcagaaagatagccggtatggctaacgtccccacttgagtgtgcagggggagatttgttgtgcggtgcccactcagtgggacccacactttgttgaaaatagaagatggtaggtgatcaaccacatgtagtttaaaaaaaaaaaaaaaaaaaagaaaaagtctttggtaggtgatccgttggtaggtggacgcgttaatcacgcatccacctctcctaatcaagcttccacctctcctataaataggagatggtgctagagcttcaatacaccaacaagcagagcaacgcagagagagattgagagagttttgagttttagaattgggttctaaaacttgggtagttaaaatagtgaagataatttagggtgtttttgggaaacacttttgagtgccactattttgtattactctctttgtaagcctactatttttaatagtggaagaaatattcggtttttgtcccgtggacgtagccctaaacagttaaagggtgaaccacgtaaatatctgtgtgtcatatttatatttttccgctgtgttattattctcgtaattttattgccgggccctaaca

SEQ ID NO: 50 - DNA, 6652 bp, Jatropha sp.
tgttggatttaattttctattgtgggcttaatatgtaaaattaaatatgtacagcagtccaattgtaaagtcaatttcaaatgtggtcttgattgtgaataattgactattgaggtatcaactaattatagatattaagtgtagagaccattatataattcctatttcgttaaaggaccgaaataggaattgtactaaccctacaggtatataaaggtcgtggtccccagaggagttgaatacgattcagttttacgttcctttcgtactgcttccgctaaatcgattcttcaattctctggaaggctgcaaacctaattctacacatcaagatcgacgaacaacgatgtcaggtacgcaaccttttcttattcaatctattttgttgaatctagtggtttgcacgatctaggttattgtggaattatttgagattaattcttaacaagtggtatcagagccttgtgcataattgctagattttagattgattttcgtatgttttttttttatatttgtttaccctactgtttacggattgagggttttgtttaataaaaaaaaaaaaaaaatttttttgttctcactgttcttcggattccccaagaacaggtaagaggttttgaactaacgccattgacgattaattgttcgtttggtattttttttttttttttttctttgctggccggatcttgtttaaaaaaaaaaaaaaaaaaaaaaaaacctccattgttggcggcggaaaggggctggaacctgttattttccgttaatcgtttttgtttttaaggttagggatggaaaattacagttaggtccctggaaaatttcgtattaggtccctaatttttctgtaacttcagattaggtccctaattttctgtaacttcagattaggcccctaaattttctgtaacttcagattaggcccctaaatttctgtaacttcagattaggcccctaaatttctgtaacttcagattaggcccctaattttctgttttctttcgtatagacccatggacatatatttaattaaattagaccccgaactttatttattcaaatttatttagtccaattttataacttagtctctaatttagtgacttaaagactagattataatttttttttgggctaaataattttagattattaaatatttagacaatcaattcatttaattgcaattgttttgttttaattacaattaaatgatttaatttgtcgacttgagattaaattttggagtgattaagtccctattgtgtttgatatttgttaaaataaattgatttgatcaataagtcaccaaagtgacctctattgcttgaattgatttattttagaatattagacaggtgtattagaattcgttgcggatagttaatcggcccaaaggaaggttgctatctggctgagttttgaatacatgtttggcaataaatatgtgataattgtttgattattttccatgacaatgatatatcggcccaaaggaagatttattatttgtcaggactatgttatcaatatttgattgctgcgttgagatactaattacaagttaatgttttacatccaaagatgagaatattaatgttgtgtctgtatcttatttccattttttgatctctacttatgtgaattgtttcatttgtggattgtgagcttatacttatctatttgtttattttctctgatgtcagctaacatgtcatttaatcctgattgtgtccctaaacttgagggatcaaattttgagtcttggaaatcagacattatgatctttctgggctgtatggatttggatcttgcattcaggatggacgaacctgctcctcttactgatacgagcactgatgttcaaagggttcagtatgagaagtgggagcgttctaatcgcttaggtctgatgatcatgcagaaaggagtcccagaacatgttcgagactctgtctcagctgaaaagaacaatgctcaaaactatcttgccgagctggagagggcttatgtcaagaatgacaaagccgaagtgggaacacttttaggaaaattaacctccatgaagtttgctggcaatggtggtgttcgggagcatattcttagcatgtctcagattgcaaagagactgaaggcgctgggtatggctttaccagatgaccttttagtccatttggttatgaattctcttccttctcagtatggacaaattaggacttcctataattgtcagccgaataaatggactcttaacgagctcatttctcattgtgttgatgaggaggaaaagatgaattctaacaaaagagagagtgctcattttgtttcttcagctgggaagggcaagaacaagaaaagaaagaggccttctgcaaagactgttgaaaaggctattgagaaacctgctgctgctactgctgtagatccagtacaaaagaagccttatgtgccaaaatgcttcttttgtggaaagacggatcacttgagaaaggactgtcctaaatttgctatttggcttgcaaagaggggtatgtaccttgttttagtctgttgtgaagttaatttaacttcagctcctaaaaacacttattggatggattctggtgctaccgctcatataagtgtttcattgcagggctgcctcaactacagaaagccaactgacggtgaaagatacgtctatgtaggaaatggcgatctggttgaagtagaggcaattggacattttagattatgtttaagtactggttattttttggatcttatagatacttttgttattccctcttttagaaggaatttgatttctgtttcctttttggacaaatctggttttcattgttcttttggagacagtgtattcactttatctatgaattcaaaagttttgggtactggttgcttgaatgaatgtgataatctatatatgttagacattgatactagttatcatgaatccctgcatgttactacaagaggtaccaaaagaaagttgaatattgataactcagctgtactttggcatagacgtttgggtcatgtatccaaatcaagaattgagcgtcttgtttctgatggtatcttagatacacttgattacacaggtttggattcttgtattgagtgtattaaaggaaaacagactaaacaaaagagactaggtgccattcggtcatccaatgttttagaactgattcatacagatatttgcggtccattccatattgcagctagaaatggccaaagatattttatatcattcattgatgactattctcgttatggttacttatatctcattcatgaaaaatctgagtctttggacgtgtttaaatcttataaagccgaagttgaaaatcaactaggaaagaagattaaatctgttcgatctgaccgtggtggtgaatattatggtaaatatgacggttcaggcgaacaacgtccaggaccttttgcgcttttcctacaggaatgtggaattgtccctcaatatacaatgcctggtactcccagcatgaatggtgtatctgagagacgtaataggactcttatggatatggtaaggagtatgattagtcattctactttaccagagtctctgtggggtgaagcgttaaagacagcagcttatcttctcaataggattccatctaaagcagttgctaaaacaccttatgaactttggactggacgtaagcctagtctaaagcatcttcatgtttggggatgtccagctgaagcaaggccttacaagccgaatgaaacaaaactggactccaaaactatcagcagctactttgttggatattctgagcggtctaggggttacaagttttataatcctacttccagaactttttttgaaacgagaattgcaacattttttgaggatgttgattttggagggagaaataaagaaagaaacattgtctttgaagaggaaccaagtaaggatgattcggttctcattccttttgttgtttctgatagcgtacagactccagttgctcctgtcgctattcaagatccaattcaagacaatgctattcttcctcaagaacaaattcaacaacctcaagaattagagcctgttcaaatgcctttgaggagatctactagggagagaagaagtgctattccagatgattatatagtgtttcttcaggaacatgaggatactgtagacatgatagatgatgatccagttagtttctatcatgccatgcagagttctgactctcataagtggattgaggcaatgaacgaagagtataagtctatgcaagacaatcaagtttgggatcttgttcaattacctgaaggaaagaaacccattggttgcaaatggatatttaaaaccaaaagggattctcaaggtaatgtggaaagatataaagcacgtcttgtagcaaaaggctttactcaaaaggaagggattgactataaagaaaccttctctccagtttcttcgaaggattcttttaggacaatcatggccttagttgcaaatcttgatcttgagcttcatcaaatggatgttaagactgcgtttctcaatggcgacattgatgaaacaatatatatggtgcaaccagaaaattttgtaattggagatccaaaacatctggtatgcaaattaaagaaatccatttatggactcaagcaagcttctcgtcaatggtactttaagttcaaccaagtaattgtctcgtttggctttgaggcaaatgctgttgaaaattgtgtgtaccataagttcagtgggagtaggtttatctttttggtcttatatgtggatgacatacttcttgctactaattgtaaagtcttgctgcatgaaaccaaagtttttctttcaaagcattttgagatgaaagatttaggtgaagcttcttttgttttagggattcagatacatcgagaccgttctcgaggtattcttggattatcacaaaagagctatattgataaagtgctcaagagatttagtatgcagaattctagaccattggatacccctattgcaaaaggtgataaatttagtaatcaacaatgtccaaagagtgaacttgaaattcaggaaatgcaaaaggttccttatgcgtcagctgtaggaagtcttatgtatgttcaagtttgtactcgtccagacatttcttttatagttggcatgttaggtagatatctcagcaatccaggaatggatcattggaaagcagccaaaagagtcatgagatatcttcagaggactaagggacatatgctcacatataggagatcagatcagcttgagatcattgggtatactgattctgatttcgctggatgccaagacagtagaaaatccacatctggctacgtttttctattggctggcggtgctatttcttggaagagtgtgaaacaaactctagtagcaacctccaccatggaagctgaatttgtagcatcttatgaggcatccaatcaagctatttggctgagaaaccttgtcactgggctgcgtgtagtaggtggaatagaaagaccactgaagttgtattgtgacagcaggtcagccgttttattctccaacaacaacaggagctcttctcgatcaaaacacattgagattaagttcttggctgtgaaggagagggtacagagtggacagatatccttagagcatattggaacaaactctatgattgcggatccgcttacaaaggcagtgataccgaaagttttccatgagcacaccgctcatatgggtatcgttttgtttgagactgtctagttttagtgggagtttgctctttatgtatacattatgtttttgaatgctctattttgttttggatatgttttattatgaaattttgttgagtactcattttggttgatggttacactctgtattattattattgttttgcttgatctcacttttgttggtgaccagttggaaacagacatgatgatcacattgcatgtagttttcatgctacacttctataatgaatctatgtcatttaggggtattggtaatgtagttgatgatgaacttagtcatgagatctatggcgaatgtttcttcagctctttatcaatataactaatggacgagattgtttttggataccttgtatgactgcgtttgttgagctcattttggtgtatgtattttatttaacatatgttcgcccaagtgggagattgttggatttaattttctattgtgggcttaatatgtaaaattaaatatgtacagcagtccaattgtaaagtcaatttcaaatgtggtcttgattgtgaataattgactattgaggtatcaactaattatagatattaagtgtagagaccattatataattcctatttcgttaaaggaccgaaataggaattgtactaaccctacaggtatataaaggtcgtggtccccagaggagttgaatacgattcagttttacgttcctttcgtactgcttccgctaaatcgattcttcaattctctggaaggctgcaaacctaattctacacatcaagatcgacgaacaacgatgtcaggtacgcaaccttttcttattcaatctattttgttgaatctagtggtttgcacgatctaggttattgtggaattatttgagattaattcttaaca

SEQ ID NO: 51 - DNA, 4984 bp, Jatropha sp.
tgtcggatgaagagaaaaagtagggatcagaatcatagttcatcatttccctgtccactaacctgtacacacagtaaaagtaaaaatcatgttaggtggaactttaggagtaattagagccgtaattagtggatagttagaagcagtttgtatattaaaacaatgtaatctttacagaaaaaaaataacaatttttccctctgtgaaacatggtatcagagctggtgtttttcagtcagaactgactgaacacatttagctcatcacctaccttctgacttacctgaaataaccaaatatcatcatgtctgatgatagtgttcatacctcgggtctcaacgacgaagacctgacaagaattgcaaacctgattaatacccaaaaccagaaacaaaccatttcagataacctcaccatcaatgtcaaattaaatagccaaaattatgcaatatggtcacgtatgatgaaagtagctattgggggaaaatcaaaaaaattgttaaaccacatcactgaagctgctacacctccatccgctggagatccacattttgaaaagtgggaacaagaagatttgattgtgttctcatggttaattcagaacatggaaccacaactagcaaacaacttaacagagtattccacagctaaagatttgtggaatgctcttgttatcacatatagcagtgggaaagataaattacagatatttgatctacataccagagcaaacagcatgaaacagggatcatcaacactagaagaattctggctcaccatgcaaggaatatggggggagatcgatcgcagggagcctaatccgatgacatgctcaacagatatcgcgacatacaacaaagtcaaacaagaacaaaaactatttcaattcttaaatggaatagatcacctgtacgatcagatcaaaagggaaatcctcagatcggagcatcttccctcagctgaagccgcctacgcctctgttcggaaagaagccgcccgattgaacatcatgggaccagctaacagagaatcgctatcacagggcataggcgacggatttgtcatcatcgggaagaaagaagcaagtgaagcaactggtgtaggactcgtcgcgcgtggccagcggcggtcagaaccaagaaacgatggttcatcatcccgtccagataaatcgagactcaagtgctcttattgtgggatgagcaaacacacaaaagatcaatgcttcgaattaattgggtatccagaatggtggaacgagaatcaccgaaacaagggcacaaagaccagcaaagcagccgccgccgtgggtaacctagaggctgccagcagcggtggcgacagcagaggaggtcagaatgagagaggagcgatggggatggtggcggcacaaaatgaaagggcagatggaaccctagaaggtgaaggaaaaagaatggggtcatttaacataaaaccccaaggaaagtatgagaaatgccccaaatcttttgaaaataataataaacagacaagaaaagataatcgctggatttttgactgtggtgctactgatacaatgacttttgaggcggctgacataatagcaccattaaaaccaaaaaaaacccatgtccaaactgcaaatgggagcactgcaaccgtagaaggagcaggatctgttgaaatttcaccgacattaaaaatatcaaattgtctttatgttccctcattgtctcataaattgttgtcgattagtcatgtgacaaaggaattaaattgtactgtactaatgcatcctaccttttgtattttacaggatatcaggagcgggaagattattggacgtggcactgagcatcatggattgtattatgtggatgaagtggcccaacacggtaatgtaatgctggctcacggaactactgaacgggaagcttggctttggcaccgacgactgggacatccatccataggttatttaagtctgttatttccaaatatatttaagtcaaataatatgtttaagtgtgagacgtgtgttttggcaaaaagtcatagacaaacttttaaaccgagtaatacccatatggagtctcctttttctttggttcattctgatgtgtggggtccagctcctgtcttaggaggtcaaggttttagatattttttactctttgttgatgactgcactcgtatgacatgggtgtattttttgaaaaataaatttgaagttgttgataaatttattctgttttatactatgatccaaactcagttcaatcaatcaattaagattttacgatctgataatgggggtgaatacataaatgaatccatgaagcaattttctcaccaaaaaggacttgtccatcaaacaacttgtcctcacacacccgaacaaaatggtgtggcagaaaggaaaaatcgcattcttcttgaaatgactcgtgccatgttaattgagtctagtgtccctagatttttttggcctgaagctattgccacagcagtctatcttttaaacaggcttcccactaaagttttaaaacttcagacaccaatacaaatgcttgctcaatttacctcaattccctcaactcttacaattcaacctcgaatatttgggtgttctgtttttgttcatatacccaaagttgaccgaacaaaatttgatccttgtgctgttaaatgtgtgtttgtgggatatggggtcaatcagaagggttatagatgttacgacccaaaaacccgtcgagtatttactaccatgaattgtgacttccttgaaactgagtattactataatacccatcccagtggtcagggggagagagaacttaaagacacactgagttggtttccaagtcaggacaatgttcaggacaacgttccatcagaaccagttaacagtaccaccgggccatctgaagcatttatacagtctacggaactcaataccactgaagaaagtcttccttcctccctgatacctgaggtaagagattctgatgctccagaaactcctgttattgaactgcctaactgtgataatttggagacctctgtagaacaggaagagaatgttggacagtatgtcctaccacccagaagtaacagaggagttccacctaaaagatattctccagaaaaagaagcacgaggaacgaaatatccagtgacaaatgtggtcagggggagcttatcaaaaagggccagagccttcactatagctctatattcagaagaagttcctcatactatagagcttgcgttaaaatcaaaaaattggaatcaggcaatgaagacggagatggatgctctaattaaaaatgacacatgggaaaaatgtcagttaccagatggaaagaaatcagtgggttgtcgttgggttttcacgatcaaacacaaactggatgggactgttgaaaggtataaagctcggttagtagcaaaaggatatacacagacgtatggtatagactattcagaaaccttctctccagtagctaaaattgatacgatcagagtattattctcgatagcagctaataaaggatggccactccatcaattcgatgtaaaaaatgcctttttacacggagatctaaaagaagaagtttatatggaagcaccaccagggttcacaaatggatttaaaactggagaagtctgtcgtttgaagaagtcactttatggtcttaaacagtctccaagagcttggtttggaagatttacaattgtaatgaagaaatatgattttaaacaaagtaattcagatcatacattattccttaagcaaaggggtaatcttattacttgtttaattatctacgttgatgatatgattattacaggaaatgatgatgaagaaatgtcaaagctgagaaggaacttattcactgaattcgaaatgaaagacttaggagcactgaagtacttccttggaattgaagtattgagatcaacaagtggaatttttatatgtcaaaagaagtatactctggatttcttggcagaaataggaatggttgactgcaagccagctgacacgcctattgcaacaaatcatggacttcagatagtagaaggagcagaatcggttgataaagaaaggtatcaacgactagttggaaaattaatttatctttcacacactcgaccagacatagcatatgcagttggagtagtaagtcagttcatgcacagacctcaaactgatcatatgaatgcagtgttaagaatcatcaagtacttaaagggcacgtctagtcaaggaattctgtttaaacggaatgggcacttggatatacaaggttatactgatgcagactgggcaggtagtcatgttgatcgcagatctacttcaggctacttcacttttgttggcggaaacttagtcacatggagaagtaagaaacagaaagttgtagcattgtctagtgcagaggcagagtttcgaggaatagctcgtggtctagctgaaattctttggctaaggaaattgttaagtgagattggttttcctccatctggagcaagtaaaattatgtgtgataatagagctgcgatcagcatttcagaaaatcctgttcaacatgataggaccaagcatgttgaaattgatagacacttcatcaaagaaaaactagaagcaggaattgtggaattgccgtttgttcgatctgaagaccagttggcagatattttgacaaaagcagttaatggaaattcctttcataacgtattgaacaagttaagtattggagatcccactactcaacttgagggggagtgtcggatgaagagaaaaagtagggatcagaatcatagttcatcatttccctgtccactaacctgtacacacagtaaaagtaaaaatcatgttaggtggaactttaggagtaattagagccgtaattagtggatagttagaagcagtttgtatattaaaacaatgtaatctttacagaaaaaaaataacaatttttccctctgtgaaaca

SEQ ID NO: 52 - DNA, 5018 bp, Jatropha sp.
tgttaaggattagttgtaaatagaactctattaggagttgtaaatagaactctattaggagttagattcctaataggtttatagtatcttgtatgtgtatatatatgtgtccctattgatgaatcaaatacagaaaaatattttcccacattaaatctacatggtatcagagcctttggctcgttaaactttttttttttccgtttgcgttgaaattccagcggtccggcaaaatccgttcatcgcggctgtcagtctaggtgaccgttctagggtcaccgttttgtctcaccgcctaccacaggaggtgcaccagctgccgatcggcgttccctccaaatccggtcaccggattccctgcgcgtgggccccacgcgccgcttcagtatttctgctacagtaccacgcgccggcgcgtggcggcgcgtccggctgtttccggcgatcggaaattgttttctggcttcgtttctacgtcagcagtctactcaagtggttgtttttccgatccggcgaagttttggtaggggcgttctcagtttttgtcaatttggcgtttcaggttacgttttgctcctgtttgcttttgaaaggctatttttttttcgtgtctctttctgcgttgctggtagggtgtttagtttttattaataatggatcctccacctacagattctaaaactactattactgaattgatcccagcaatgaataaaatcacagaacataaattgaaaggaactaattatttagattggagtaagacaattcgaatttatttgcgaagtgttgaaaaagatgatcatctaattgatgatccaccaactgatgacactaggcaacgttggctcagggaagatgctaagttatttctactgattcgcaattctattgacagtgaagtggttggattaatcaatcattgtgagtttgttaaagaattaatggaatatcttgaattcttgtactcgggaaaaggtaatctgtctcgtatgtatgaggtctgcaaaggattttatcgggccgaccagcaagataagccccttacagcttatttcatggaatttaagaagatttacgaggaacttaatgttttattgccttttagtcccgatgttaaggttcaacagacgcagcgagaacaaatggctgtgatgagttttcttgcaggtcttgctccagtttttgaaactgccaaatcacaagttctttcaggcagtgatgttacctctcttcaggagacttttgctagattgctacgtactgaaggttctcatacaccttccccttctgataatagtgcccttgtgagtcgtaatcaggtagctagacagccaggaaacagaggaagaggaggaaataaccgtagtaatggaccaatcactcgggagacaagcattcttatctgccattattgtcatgaacctggtcacactaaacggtattgcaagaaactaataaataagccaccaagggtaaatatggcacatcaagattatgcagttacttcttctgacaacaatgtaacaatttctgctgcggagtatgctgagctctctagataccgttcctctaagtctactgattcctcaattgctgctgctgccaactcaggtactcctccttcctgtcttatgtctacctcatcagagtgggtcattgattctggttgcaccgatcacatgacaggtaaccctagtctcttatcctcattaaaagaacctaaacaatttaatcgtcatgttactctagctgatgggtccacatctcctgtcaagtgtattggcactgccaatcccattccatctcttccattgtcttctgttttttgtttgcctcgtttttcgtttaatctattatctgttagcaaaattactcgtatgctgaattgttgtgtcaaattttatcctacatattgtgtgtttcaggatcttttgacgaagcggattattggtagaggacgtgagtctggtggcctttatcttctggatgaacacgtttcattatctctggcttgtgctggtgctctatctcctcttgaggttcattgtcgtttgggtcatccttcattgtctactttaaagaagttgtgtccgcagtttcagtctttatcaagtttagaatgtgaatcttgtcaatttgccaaacatcatcgtttacctttagttcctcgagtcaataaacgggtttcatccccttttgagttagttcattctgatgtttggggtccatgtcctgttatttctaaaactggctttcggtattttgtaacttttgtggatgattattctcgtgttacttggttatttttaatgaaaactcgttctgagatattctctattttctgttctttttgtgctgaaattaaaactcaatttaatacttctgttcgcatattgcacagtgataatgctaaagaatacctatctagttccttccaatcttatatgtcacaaaatggtattctccatcaaacatcttgtgttgatactccttctcaaaatggggttgcagaaagaaagaatcgacatcttcttgaggttgctagggcacttttattccaaatgaaggtccctaaacatttttgggctgatgctgtctctactgcatgttttttgattaatcgcatgccttcttctgtgcttaatggtgcaattccttataatgtcttgtttccttctaaacttctattccccattgaacctcgtatttttggttgtacttgttttgttcgtgatgttcgtccacaggtttccaaattagatccaaagtcccttcgttgtgttttccttgggtattctcggcatcaaaaagggtatcgttgtttctctcccgatcttgaccgttatcttatttctgcagatgtcacattttttgagaatactccattttttccttcttctatatataagagtgagggggaggatgatgaccttttagtctatgctatccagcaagttcataactctcctgctcctgctcgtattcctgaccttgctcctactcctgctccagcgtctgctgctcctattcccgctcctgcaccaacatctacacgccctcgttttgaacaggtttattccaggagaccaggaactcttgactcacttcctccacctgccgtttcaacagaagatccggttctctccactcaacctgagcctgtgccatccactccccagtcagaccttgaccttcccattgctcttcgaaaaggtaaacgcaagtgtacctaccctatttcctcttttgttagttatgatcatctttcttcttcatctcgttgttttgtaagtaacttagactctgtctctattcctaaaactgttgtccaagctttgtcccatcctggttggtgtgcagcaatgaaagaggagatggctgccttagatactaatggtacttgggatcttgtgcctctcccggcaggtaagaaagctattggttgtaagtgggttttcaccgttaaagtgaatcctgatgggactgttgctcgtctcaaggctcgattagttgcaaagggctattctcagatctatggccttgattattctgatactttttctcctgttgccaaattgtcttctgttcgtttatttatttctttagcagctacttttgactggcctttacatcagctggacgttaagaatgcctttcttcatggggatcttcaggaggaagtttacatggagcaaccaccaggctttgttgctcagggggagtcgggtaaagtgtgtaaacttcgaaaatctctttatggcctaaaacagagtcctcgagcttggtttggtagattttgcaatgtggtacatcaatttggcatgcagaagagtacttctgatcattctgtatttttcagaagtactaagactggtttgattctacttgtggtttatgttgatgacattgttattacagggagtgattcagccggcattacatcactcaagtcatttctccagacccagttccagacaaaagacttaggattgttgaagtattttttgggtattgaagtatctaggtgtaagagaggcatcttcttatctcaaaggaagtatgtccttgacttactgaaggaaactggaaagttgggtgccaagccatgcagtgctccaatgactccaaatttgcaactttcaattggtggtgagctatttgatgatcctgagatgtatagaaggttagtgggcaaattgaattatcttaccgtaacacgtccagatattgcttactctgtgagcgtcttgagccagtttatgtcttctcctactgttatccattgggaagctttaggacagattttgtgctacttgaaaggtaatccaggtcgtggcctgctttacagtaatcataggcatttgaacattgaatgtttttcagatgcggattgggcaggttcgaaggttgatagaagatcaacttcaggatattgtgtctttgtaggtggaaacttgatatcatggaggagcaagaaacaaaatgtagtctcacgatcgagtgctgaatcagaatatcgagctatggcacaatctgcttgtgaattaatttggattcgtcagttgttagaggaaattggcttcaagtcatccttgcctgcaaagttatggtgtgataatcaagctgctcttcacattgcatcaaatccagtattccatgaacgaacaaaacatatcgagattgattgtcatttcattcgtgaaaagatacaacagggtgttatctctacaggacacgttaaaacaagtgaacagttaggggatatctttacaaaagctctaaacggaccccgggttgactatctttgtaacaagttaggcatgattaatatctatgctccaacttgagggggagtgttaaggattagttgtaaatagaactctattaggagttgtaaatagaactctattaggagttagattcctaataggtttatagtatcttgtatgtgtatatatatgtgtccctattgatgaatcaaatacagaaaaatattttcccacattaaatctaca